(19) United States
(12) Patent Application Publication
(10) Pub. No.: US 2019/0124314 A1
(43) Pub. Date: Apr. 25, 2019

(54) THREE-DIMENSIONAL STATE ESTIMATION DEVICE, THREE-DIMENSIONAL STATE ESTIMATION PROGRAM, AND THREE-DIMENSIONAL STATE ESTIMATION METHOD

(71) Applicant: NATIONAL INSTITUTE OF ADVANCED INDUSTRIAL SCIENCE AND TECHNOLOGY, Tokyo (JP)

(72) Inventors: Takao Ueda, Ibaraki (JP); Tatsuya Oki, Ibaraki (JP)

(21) Appl. No.: 16/093,557

(22) PCT Filed: Mar. 9, 2017
(86) PCT No.: PCT/JP2017/009465

§ 371 Date: Oct. 12, 2018

(30) Foreign Application Priority Data
Apr. 14, 2016 (JP) 2016-080891

(51) Int. Cl. H04N 13/106; G06T 7/50; H04N 13/275; G06T 7/60; G01N 33/24

(52) U.S. Cl. CPC H04N 13/106; G06T 7/50; G01N 33/24; G06T 7/60; H04N 13/275

(57) ABSTRACT

To estimate a three-dimensional state of an internal structure of particles forming a multicomponent material containing unknown materials based on two-dimensional image data, a three-dimensional state estimation device according to the present invention comprises statistical data setting means for setting statistical data obtained by taking statistics of a correlation among: a complexity indicator for quantitatively indicating, as an image complexity, various display states of a component of interest in two-dimensional image data in which a cross-section or the like of a multicomponent material is displayed and the component of interest and a component of non-interest in group of particles in the cross-section or the like are displayed differently from each other; an area fraction of the component of interest in the two-dimensional image data; and three-dimensional estimation data, which is, for example, three-dimensional state data on a content percentage of the component of interest in the multicomponent material at a time when the complexity indicator and the area fraction are determined.

### TECHNICAL FIELD

The present invention relates to a three-dimensional state estimation device, a three-dimensional state estimation program, and a three-dimensional state estimation method, which use two-dimensional image data on a multicomponent material containing a group of particles formed of liberated particles and locked particles to estimate a three-dimensional state of an internal structure of the multicomponent material.

### BACKGROUND ART

A group of particles obtained by crushing a multicomponent material, for example, a natural ore, contain a mixture of a particle formed of a single component and a particle formed of a plurality of components.

A state of a particle being formed of a single component is generally referred to as “liberation”, and the particle formed of a single component is generally referred to as a “liberated particle”. A value indicating a percentage of the liberated particle of a specific component contained in the multicomponent material, which is obtained by, for example, dividing the mass of the liberated particle by the mass of the specific component contained in the multicomponent material, is generally referred to as a “degree of liberation”.

Further, the particle formed of a plurality of components is generally referred to as a “locked particle”, and a value indicating a percentage of the locked particle contained in the multicomponent material is generally referred to as a “degree of locking”.

It is important to accurately measure the degree of liberation of the generated crushed material in order to efficiently retrieve a useful metal from, for example, a natural ore.

For example, the useful metal can be retrieved at a high proportion from a natural ore having a high degree of liberation of the useful metal, whereas, for example, the useful metal is retrieved at a low proportion from a natural ore having a low degree of liberation of the useful metal, resulting in a large load on processing of sorting out the useful metal from, for example, the natural ore. Therefore, in the processing of developing, for example, a mine from which to produce, for example, the natural ore, it is important to accurately measure the degree of liberation of the useful metal in advance, and optimize a crushing/grinding method while accurately estimating the quality of, for example, the mine.

As a method of measuring the degree of liberation of, for example, the natural ore, there has hitherto been generally performed a method involving cutting a sample obtained by hardening a target particle with a resin and observing a two-dimensional particle structure of the sample, which is observed from the cross-section thereof.

However, the method of observing the two-dimensional particle structure causes a bias of misunderstanding that the target particle is the liberated particle depending on a position of a cross-section to be cut although the target particle is the locked particle. This bias is called “stereological bias”, and the above-mentioned method cannot avoid this bias in principle. As a result, the degree of liberation is known to be overestimated.

Now, a description is given of a situation of occurrence of a stereological bias with reference to FIG. 1. FIG. 1 is an explanatory diagram for illustrating the situation of occurrence of a stereological bias.

As illustrated in FIG. 1, a target particle P1 is a locked particle formed of a component A and a component B. When the cross-section of the target particle P1 at a position X is observed, the target particle P1 is misunderstood to be a liberated particle of the component A. Further, when the cross-section of the target particle P1 at a position Y is observed, the target particle P1 is understood to be a locked particle of the component A and the component B. Further, when the cross-section of the target particle P1 at a position Z is observed, the target particle P1 is misunderstood to be a liberated particle of the component B.

That is, in the method of observing the two-dimensional particle structure, in a case where the target particle P1 is the locked particle and its cross-section is observed at three positions, the target particle P1 is correctly recognized to be the locked particle only when its cross-section is observed at the position Y, whereas a stereological bias of causing misunderstanding that the target particle P1 is the liberated particle occurs when its cross-section is observed at the remaining two positions X and Z. Further, this stereological bias inevitably causes the degree of liberation to be overestimated based on misunderstanding caused in the observation of the cross-section at the positions X and Z.

As a method of reducing misunderstanding caused by the stereological bias, there is proposed a method involving measuring the number of particles apparently liberated from one another to obtain the degree of liberation based on observation of the cross-section of the multicomponent material, and dividing the degree of liberation by an experimental coefficient called a “locking factor”, to thereby predict a true degree of liberation (refer to Non-patent Documents 1 and 2).

However, the locking factor used in this proposition is set without consideration of an influence of the internal structure of particles, and thus there is a problem in that, for example, this method cannot be applied to multicomponent materials other than a multicomponent material used in the experiment.

Further, as a method of reducing the risk of misunderstanding caused by the stereological bias, there is proposed a method involving measuring a content percentage of a component of interest for each target particle from observation of the cross-section of the multicomponent material, setting the content percentage as the two-dimensional degree of locking, and converting the two-dimensional degree of locking into the three-dimensional degree of locking by an experimentally obtained kernel function for correction (refer to Non-patent Document 3). In this proposition, a particle having the degree of locking of 0% or 100% of the component of interest corresponds to the liberated particle.

However, there is a problem in that the kernel function used in this proposition is a function intrinsic to the multicomponent material used in the experiment, and cannot be applied to multicomponent materials other than the multicomponent material. Further, in the process of an experiment for obtaining the kernel function, the three-dimensional degree of locking is obtained, and thus there is a contradiction that the two-dimensional degree of locking is not required to be converted into the three-dimensional degree of locking for correction. That is, this proposition is not a method of estimating the three-dimensional state of the multicomponent material other than the multicomponent material used in the experiment.

### PRIOR ART DOCUMENTS

### Non-patent Documents

Non-Patent Document 1: M. Gaudin, A., Principles of Mineral Dressing, 1939.
Non-Patent Document 2: B. Petruk, W., Correlation between grain sizes in polished section with sieving data and investigation of mineral liberation measurements from polished sections, Trans. Inst. Min. Metall. Sect. C. 87 (1978) C272-C277.
Non-Patent Document 3: R.P. King, C. L. Schneider, Stereological correction of linear grade distributions for mineral liberation, Powder Technol. 98 (1998) 21-37.

### SUMMARY OF THE INVENTION

### Problems to be Solved by the Invention

The present invention has an object to solve the above-mentioned problems in the related art, and provide a three-dimensional state estimation device, a three-dimensional state estimation program, and a three-dimensional state estimation method, which are capable of estimating a three-dimensional state of an internal structure of particles forming a multicomponent material containing unknown materials based on two-dimensional image data on the multicomponent material.

The inventors of the present invention have conducted an extensive investigation in order to achieve the above-mentioned object, and the following knowledge has been obtained.

First, it is assumed that two components of a target particle (locked particle) exist as illustrated in FIG. 2. FIG. 2 is an explanatory diagram for illustrating an example of existence states of two components in the target particle.

A target particle P2 illustrated in FIG. 2 has the component B as its base material and the component A is spread in the particle unlike the model illustrated in FIG. 1. The amount of the component A contained in the particle is equivalent to that of the model illustrated in FIG. 1, but the target particle P2 can be recognized to be a locked particle of the component A and the component B even when its cross-section is observed at any of the positions X, Y, and Z. Thus, a frequency of occurrence of misunderstanding caused by the stereological bias can be said to be smaller than that of the model illustrated in FIG. 1.

A reason for the difference in frequency of occurrence of misunderstanding caused by the stereological bias between the model illustrated in FIG. 1 and the model illustrated in FIG. 2 resides in existence states of the component A and the component B in the target particle. Specifically, in the model illustrated in FIG. 1, the component A is contained in a fixed region of the target particle P1 as one mass in a dense state, whereas in the model illustrated in FIG. 2, the component A is spread in the target particle P2, that is, contained in the entire particle in a sparse state.

Further, the difference in state of the component A between the target particles P1 and P2 can be observed from two-dimensional data such as the cross-section or surface. This means that it is possible to obtain three-dimensional data on the existence states of the component A in the target particles P1 and P2 based on how the component A exists in, for example, the cross-sections of the target particles P1 and P2, which is observed from the two-dimensional data.

Thus, the inventors of the present invention have examined whether the fact that the display states of a specific component of, for example, a useful metal displayed in two-dimensional image data on, for example, the cross-section of an ore, are not uniform, that is, diverse, can be used to estimate three-dimensional state data on the percentage of the specific component contained in the ore from the two-dimensional image data by using an indicator for quantitatively indicating the diverse display states in the two-dimensional image data as the complexity of the image.

As a result, it is found that, when statistical data that uses the indicator for quantitatively indicating the diverse display states of a freely-selected group of ores in the two-dimensional image data as the complexity of the image is set in advance, the three-dimensional state data can be estimated surprisingly accurately by collating a display state of an unknown ore with the statistical data that uses the indicator.

Further, the three-dimensional state data is estimated by using the fact that the display state of the specific component in the two-dimensional image data is not uniform, and thus the estimation target is not limited to an ore, but can be applied to a multicomponent material whose particle contains specific components in various existence states as illustrated in FIG. 1 and FIG. 2. For example, it is possible to estimate even the three-dimensional state data on, for example, crushed particles of a waste containing a plurality of components.

### Means for Solving Problems

The present invention has been made based on the above-mentioned knowledge, and means for solving the above-mentioned problems is configured in the following manner.

That is, <1> a three-dimensional state estimation device according to the present invention comprises statistical data setting means for setting statistical data obtained by taking statistics of a correlation among: a complexity indicator for quantitatively indicating, as an image complexity, various display states of a component of interest in two-dimensional image data in which a cross-section or surface of a multicomponent material comprising a group of particles formed of a liberated particle and a locked particle is displayed and the component of interest and a component of non-interest in the group of particles in the cross-section or surface are displayed differently from each other; an area fraction of the component of interest in the two-dimensional image data; and three-dimensional estimation data, which is any one of three-dimensional state data on a content percentage of the component of interest in the multicomponent material at a time when the complexity indicator and the area fraction are determined and correction data for correcting two-dimensional state data on the content percentage of the component of interest in the cross-section or surface of the multicomponent material to the three-dimensional state data.

<2> In the three-dimensional state estimation device according to the said <1>, the three-dimensional estimation data used for setting the statistical data by the statistical data setting means is the three-dimensional state data, and the three-dimensional state estimation device further comprises first three-dimensional state estimation means for deriving, when the complexity indicator and the area fraction of the multicomponent material serving as an estimation target are input, the three-dimensional state data corresponding to the input of the complexity indicator and the area fraction through collation with the statistical data set in the statistical data setting means, and capable of directly outputting the derived three-dimensional state data as true value estimation data for estimating a three-dimensional state of the estimation target.

<3> In the three-dimensional state estimation device according to the said <1>, the three-dimensional estimation data used for setting the statistical data by the statistical data setting means is the correction data, and the three-dimensional state estimation device further comprises second three-dimensional state estimation means comprising: a correction data deriving module configured to collate input of the complexity indicator and the area fraction of the multicomponent material serving as an estimation target with the statistical data set in the statistical data setting means, to thereby derive the correction data corresponding to the input of the complexity indicator and the area fraction; and a two-dimensional state data correction module configured to correct the input two-dimensional state data on the multicomponent material serving as the estimation target through use of the correction data derived by the correction data deriving module, to thereby derive the three-dimensional state data, and capable of outputting the derived three-dimensional state data as true value estimation data for estimating a three-dimensional state of the estimation target.

<4> In the three-dimensional state estimation device according to any one of the said <1>to <3>, the complexity indicator is any one of a fractal dimension value and a statistical feature calculated due to a difference in the image density value when different image density values are given to the component of interest and the component of non-interest.

<5> In the three-dimensional state estimation device according to the said <4>, the fractal dimension value δ is calculated in accordance with Equation (1) given below,

$$\begin{matrix}[ Numerical Equation 1 ] & \\ δ = 2 - \frac{log A ( r ) - C}{log r} & ( 1 )\end{matrix}$$

where: r indicates a length of one side of a defined square region, which is defined by equally dividing a square region having a length of one side being R in the two-dimensional image data into N² blocks by any integer N; A(r) indicates, when respective vertices of a square in the defined square region are denoted by A, B, C, and D, plane coordinates X and Y are set in the same plane as a plane of the vertices A, B, C, and D, and respective points set depending on image strengths at the respective vertices A, B, C, and D in the two-dimensional image data as a height Z in a direction orthogonal to the plane forming the plane coordinates X and Y are denoted by set points A′, B′, C′, and D′, a sum of areas of two triangles comprising one triangle having the set points A′, B′, and D′ as vertices, and another triangle having the set points B′, C′, and D′ as vertices, which are calculated for all the defined square regions in the square region; and C indicates log A(1).

<6> In the three-dimensional state estimation device according to the said <4>, the three-dimensional state estimation device is configured to calculate the statistical feature through use of a density co-occurrence matrix P(i,j:d,θ), which is a matrix indicating, when an entire or partial region of the two-dimensional image data represented by two or more tones of the density level is observed with XY plane coordinates, a frequency in the entire or partial region of a pair of a pixel 1 with a pixel density value of i and a pixel 2 with a pixel density value of j, which are any two pixels in the entire or partial region, where d represents a coordinate distance between the pixel 1 and the pixel 2 and θ represents an angle formed by a straight line connecting the two pixels and an X axis.

<7> In the three-dimensional state estimation device according to the said <4>, the three-dimensional state estimation device is configured to calculate the statistical feature through use of a density difference vector Q(i:d,θ), which is a vector indicating, when an entire or partial region of the two-dimensional image data represented by two or more tones of the density level is observed with XY plane coordinates, a frequency in the entire or partial region of a pair of a pixel 1 and a pixel 2, which are any two pixels in the entire or partial region, with a difference between a pixel density value of the pixel 1 and pixel density value of the pixel 2 being i, where d represents a coordinate distance between the pixel 1 and the pixel 2 and θ represents an angle formed by a straight line connecting the two pixels and an X axis.

<8>In the three-dimensional state estimation device according to any one of the said <1> to <7>, the three-dimensional state data is a degree of liberation indicating any one of an area fraction, a volume fraction, a mass fraction, and a count fraction of a liberated particle in the group of particles.

<9> In the three-dimensional state estimation device according to any one of the said <1> to <7>, the three-dimensional state data is a degree of locking indicating any one of an area fraction, volume fraction, mass fraction, and count fraction in a group of locked particles in which any one of an area fraction, volume fraction, and mass fraction of a component of interest in one particle is a fixed fraction.

<10> A three-dimensional state estimation program according to the present invention is a program for causing a computer to function as statistical data setting means for setting statistical data obtained by taking statistics of a correlation among: a complexity indicator for quantitatively indicating, as an image complexity, various display states of a component of interest in two-dimensional image data in which a cross-section or surface of a multicomponent material comprising a group of particles formed of a liberated particle and a locked particle is displayed and the component and a component of non-interest in the group of particles in the cross-section or surface are displayed differently from each other; an area fraction of the component of interest in the two-dimensional image data; and three-dimensional estimation data, which is any one of three-dimensional state data on a content percentage of the component of interest in the multicomponent material at a time when the complexity indicator and the area fraction are determined and correction data for correcting two-dimensional state data on the content percentage of the component of interest in the cross-section or surface of the multicomponent material to the three-dimensional state data.

<11> A three-dimensional state estimation method according to the present invention comprises a statistical data setting step of setting statistical data obtained by taking statistics of a correlation among: a complexity indicator for quantitatively indicating, as an image complexity, various display states of a component of interest in two-dimensional image data in which a cross-section or surface of a multicomponent material comprising a group of particles formed of a liberated particle and a locked particle is displayed and the component of interest and a component of non-interest in the group of particles in the cross-section or surface are displayed differently from each other; an area fraction of the component of interest in the two-dimensional image data; and three-dimensional estimation data, which is any one of three-dimensional state data on a content percentage of the component of interest in the multicomponent material at a time when the complexity indicator and the area fraction are determined and correction data for correcting two-dimensional state data on the content percentage of the component of interest in the cross-section or surface of the multicomponent material to the three-dimensional state data.

### Advantageous Effects of the Invention

According to the present invention, it is possible to solve the above-mentioned problems in the related art, and provide the three-dimensional state estimation device, the three-dimensional state estimation program, and the three-dimensional state estimation method, which are capable of estimating the three-dimensional state of the internal structure of particles forming the multicomponent material containing unknown materials based on the two-dimensional image data on the multicomponent material.

### BRIEF DESCRIPTION OF DRAWINGS

FIG. 1 is an explanatory diagram for illustrating a situation of occurrence of a stereological bias.

FIG. 2 is an explanatory diagram for illustrating an example of existence states of two components in a target particle.

FIG. 3 is a block diagram for illustrating a configuration of a three-dimensional state estimation device and a flow of estimation processing in a first embodiment of the present invention.

FIG. 4(a) is an explanatory diagram for illustrating a two-dimensional image.

FIG. 4(b) is an explanatory diagram for illustrating two-dimensional image data.

FIG. 5 are explanatory diagrams for illustrating a process of calculating a fractal dimensional value δ.

FIG. 6 is a diagram for illustrating, as a contour line diagram, statistical data obtained by taking statistics of a correlation among the fractal dimensional value δ, an area fraction Fa, and the value of three-dimensional state data.

FIG. 7(a) is a contour line diagram obtained by setting f₁ as a statistical feature.

FIG. 7(b) is a contour line diagram obtained by setting f₂ as a statistical feature.

FIG. 7(c) is a contour line diagram obtained by setting f₄^{r} or as a statistical feature.

FIG. 7(d) is a contour line diagram obtained by setting f₅ as a statistical feature.

FIG. 7(e) is a contour line diagram obtained by setting f₆^{r} as a statistical feature.

FIG. 7(f) is a contour line diagram obtained by setting f₇ as a statistical feature.

FIG. 7(g) is a contour line diagram obtained by setting f₇^{r} as a statistical feature.

FIG. 7(h) is a contour line diagram obtained by setting f₈ as a statistical feature.

FIG. 7(i) is a contour line diagram obtained by setting f₈^{r} as a statistical feature.

FIG. 7(j) is a contour line diagram obtained by setting f₉ as a statistical feature.

FIG. 7(k) is a contour line diagram obtained by setting f₉^{r} as a statistical feature.

FIG. 7(l) is a contour line diagram obtained by setting f₁₀ as a statistical feature.

FIG. 7(m) is a contour line diagram obtained by setting f₁₁ as a statistical feature.

FIG. 7(n) is a contour line diagram obtained by setting f₁₁^{r} as a statistical feature.

FIG. 7(o) is a contour line diagram obtained by setting f₁₂^{r} as a statistical feature.

FIG. 7(p) is a contour line diagram obtained by setting f₁₃ as a statistical feature.

FIG. 7(q) is a contour line diagram obtained by setting f₁₄ as a statistical feature.

FIG. 7(r) is a contour line diagram obtained by setting f₁₄^{r} as a statistical feature.

FIG. 8 is an explanatory diagram for illustrating a calculation region.

FIG. 9(a) is a contour line diagram obtained by setting f_{con} as a statistical feature.

FIG. 9(b) is a contour line diagram obtained by setting fₐₛₘ as a statistical feature.

FIG. 9(c) is a contour line diagram obtained by setting fₑₙₜ as a statistical feature.

FIG. 9(d) is a contour line diagram obtained by setting fₑₙₜ^{r} as a statistical feature.

FIG. 9(e) is a contour line diagram obtained by setting fₘₑₐₙ as a statistical feature.

FIG. 10 is a block diagram for illustrating a configuration of a three-dimensional state estimation device and a flow of estimation processing in a second embodiment of the present invention.

FIG. 11 is a diagram for illustrating, as a contour line diagram, statistical data obtained by taking statistics of the correlation among the fractal dimensional value δ, the area fraction Fa, and the value of a stereological bias corrected value.

FIG. 12 is a conceptual diagram of a degree of locking.

FIG. 13(a) is a contour line diagram (Λ_{A}^{3D}) in a case where a content percentage of a component A is 0%.

FIG. 13(b) is a contour line diagram (Λ_{A}^{3D}) in a case where the content percentage of the component A is larger than 0% and smaller than 10%.

FIG. 13(c) is a contour line diagram (Λ_{A}^{3D}) in a case where the content percentage of the component A is larger than 10% and smaller than 20%.

FIG. 13(d) is a contour line diagram (Λ_{A}^{3D}) in a case where the content percentage of the component A is larger than 20% and smaller than 30%.

FIG. 13(e) is a contour line diagram (Λ_{A}^{3D}) in a case where the content percentage of the component A is larger than 30% and smaller than 40%.

FIG. 13(f) is a contour line diagram (Λ_{A}^{3D}) in a case where the content percentage of the component A is larger than 40% and smaller than 50%.

FIG. 13(g) is a contour line diagram (Λ_{A}^{3D}) in a case where the content percentage of the component A is larger than 50% and smaller than 60%.

FIG. 13(h) is a contour line diagram (Λ_{A}^{3D}) in a case where the content percentage of the component A is larger than 60% and smaller than 70%.

FIG. 13(i) is a contour line diagram (Λ_{A}^{3D}) in a case where the content percentage of the component A is larger than 70% and smaller than 80%.

FIG. 13(j) is a contour line diagram (Λ_{A}^{3D}) in a case where the content percentage of the component A is larger than 80% and smaller than 90%.

FIG. 13(k) is a contour line diagram (Λ_{A}^{3D}) in a case where the content percentage of the component A is larger than 90% and smaller than 100%.

FIG. 13(l) is a contour line diagram (Λ_{A}^{3D}) in a case where the content percentage of the component A is 100%.

FIG. 14(a) is a contour line diagram (Λ_{A}^{Dif}) in a case where the content percentage of the component A is 0%.

FIG. 14(b) is a contour line diagram (Λ_{A}^{Dif}) in a case where the content percentage of the component A is larger than 0% and smaller than 10%.

FIG. 14(c) is a contour line diagram (Λ_{A}^{Dif}) in a case where the content percentage of the component A is larger than 10% and smaller than 20%.

FIG. 14(d) is a contour line diagram (Λ_{A}^{Dif}) in a case where the content percentage of the component A is larger than 20% and smaller than 30%.

FIG. 14(e) is a contour line diagram (Λ_{A}^{Dif}) in a case where the content percentage of the component A is larger than 30% and smaller than 40%.

FIG. 14(f) is a contour line diagram (Λ_{A}^{Dif}) in a case where the content percentage of the component A is larger than 40% and smaller than 50%.

FIG. 14(g) is a contour line diagram (Λ_{A}^{Dif}) in a case where the content percentage of the component A is larger than 50% and smaller than 60%.

FIG. 14(h) is a contour line diagram (Λ_{A}^{Dif}) in a case where the content percentage of the component A is larger than 60% and smaller than 70%.

FIG. 14(i) is a contour line diagram (Λ_{A}^{Dif}) in a case where the content percentage of the component A is larger than 70% and smaller than 80%.

FIG. 14(j) is a contour line diagram (Λ_{A}^{Dif}) in a case where the content percentage of the component A is larger than 80% and smaller than 90%.

FIG. 14(k) is a contour line diagram (Λ_{A}^{Dif}) in a case where the content percentage of the component A is larger than 90% and smaller than 100%.

FIG. 14(l) is a contour line diagram (Λ_{A}^{Dif}) in a case where the content percentage of the component A is 100%.

FIG. 15 is a diagram for illustrating a particle size distribution of spherical particles.

FIG. 16 is a diagram for illustrating created aggregated particles.

FIG. 17(a) is a diagram for illustrating spherical particles created by a distinct element method.

FIG. 17(b) is a diagram for illustrating a state of generation of phase A elements in spherical elements.

FIG. 17(c) is a diagram for illustrating created spherical two-component particles.

FIG. 18(a) is a contour line diagram obtained by taking statistics of a correlation among a degree of liberation (L_{A}^{3D}), the fractal dimensional value (δ), and the area fraction (Fa).

FIG. 18(b) is a contour line diagram obtained by taking statistics of a correlation among a degree of liberation (L_{B}^{3D}), the fractal dimensional value (δ), and the area fraction (Fa).

FIG. 19(a) is a contour line diagram obtained by taking statistics of a correlation among a degree-of-liberation over-estimation rate (σ_{A}), the fractal dimensional value (δ), and the area fraction (Fa).

FIG. 19(b) is a contour line diagram obtained by taking statistics of a correlation among a degree-of-liberation over-estimation rate (σ_{B}), the fractal dimensional value (δ), and the area fraction (Fa).

FIG. 20 are explanatory diagrams for illustrating particles created by a geodesic grid method.

FIG. 21 are diagrams for illustrating examples of particles each having a spherical shape as its basic shape.

FIG. 22 is a diagram for illustrating a multicomponent material formed of model particles.

FIG. 23(a) is an explanatory diagram (1) for illustrating a method of setting two components, namely, a phase A and a phase B to a model particle.

FIG. 23(b) is an explanatory diagram (2) for illustrating a method of setting two components, namely, the phase A and the phase B to a model particle.

FIG. 23(c) is an explanatory diagram (3) for illustrating a method of setting two components, namely, the phase A and the phase B to a model particle.

FIG. 24 is a diagram for illustrating 12 types of model particles.

FIG. 25 is a diagram for illustrating a cross-section of the multicomponent material created by a model particle (α=2.0, Sc=0.914) of No. 11.

FIG. 26 is a graph for showing a comparison between an estimation error of the degree of liberation (E) for a true value (L_{B}^{3D}) that is obtained by substituting true value estimation data (L_{B}^{3D′}) for the degree of liberation of an estimation target in a three-dimensional state into L_{B}^{est} and an estimation error of the degree of liberation (E) for the true value (L_{B}^{3D}) that is obtained by substituting the degree of liberation (L_{B}^{2D}) of the estimation target in a two-dimensional state into L_{Best.}

FIG. 27 is a graph for showing a comparison between the estimation error of the degree of liberation (E) for the true value (L_{B}^{3D}) that is obtained by substituting true value estimation data (L_{B}^{3D″}) for the degree of liberation of the estimation target in the three-dimensional state, which is obtained through correction, into L_{B}^{est} and the estimation error of the degree of liberation (E) for the true value (L_{B}^{3D}) that is obtained by substituting the degree of liberation (L_{B}^{2D}) of the estimation target in the two-dimensional state into L_{B}^{est}.

### MODES FOR CARRYING OUT THE INVENTION

(Three-dimensional State Estimation Device)

In the following, a description is given in detail of first and second embodiments according to a three-dimensional state estimation device of the present invention with reference to the drawings.

### First Embodiment

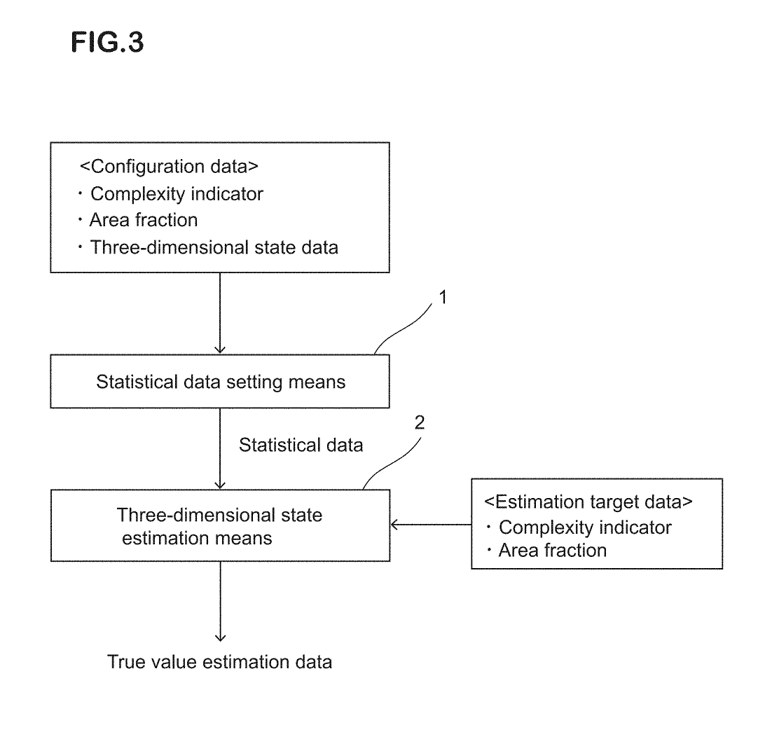

FIG. 3 is a block diagram for illustrating a configuration of a three-dimensional state estimation device and a flow of estimation processing according to the first embodiment.

As illustrated in FIG. 3, the three-dimensional state estimation device according to the first embodiment comprises statistical data setting means 1 and three-dimensional state estimation means 2.

The three-dimensional state estimation device according to the first embodiment is configured to estimate a three-dimensional state of an internal structure of a multicomponent material, which contains a group of particles formed of a liberated particle and a locked particle, based on data obtained from two-dimensional image data on the multicomponent material.

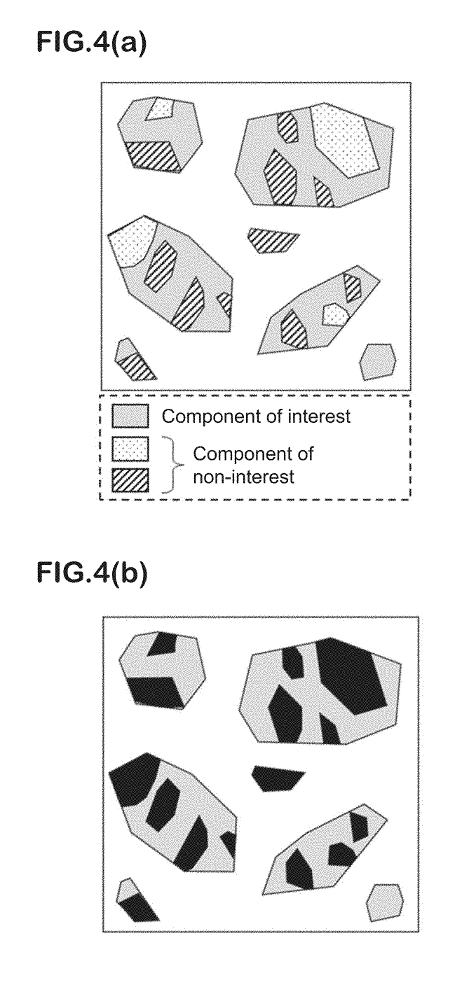

The two-dimensional image data is data for displaying a cross-section or surface of the multicomponent material in which a component of interest and a component of non-interest of the group of particles are in different display states. For example, compared with a two-dimensional image obtained by using a publicly known image acquisition apparatus such as an electronic microscope or an energy dispersive X-ray analyzer illustrated in FIG. 4(a), the two-dimensional image data is data for displaying the component of interest and the component of non-interest in different image densities as illustrated in FIG. 4(b). FIG. 4(a) is an explanatory diagram for illustrating the two-dimensional image, and FIG. 4(b) is an explanatory diagram for illustrating the two-dimensional image data.

In FIG. 4(a) and FIG. 4(b), one component is set as the component of interest among three components, but the component of interest may be freely selected from among those three components. In FIG. 4(a) and FIG. 4(b), one component among the two components displayed as the component of non-interest may be set as the component of interest.

Further, image processing illustrated in FIG. 4(b) is conducted on the two-dimensional image of FIG. 4(a) to perform subsequent estimation processing in a simplified manner, and as illustrated in FIG. 4(a), the two-dimensional image itself can also be treated as the two-dimensional image data as long as the component of interest and the component of non-interest are displayed in different display states. Further, the two-dimensional image itself may also be treated as the two-dimensional image data when the particle is formed of two components.

The type of the multicomponent material is not particularly limited as long as the multicomponent material contains the group of particles formed of the liberated particle and the locked particle, and an ore comprising a useful metal and various industrial wastes comprising a valuable material can be given as the multicomponent material.

The statistical data setting means 1 is capable of setting statistical data obtained by taking statistics of a correlation among a complexity indicator, an area fraction, and three-dimensional state data serving as three-dimensional estimation data.

The three-dimensional state estimation means 2 is capable of deriving, when the complexity indicator and the area fraction of a multicomponent material serving as an estimation target are input, the three-dimensional state data corresponding to the input of the complexity indicator and the area fraction through collation with the statistical data set in the statistical data setting means 1, and outputting the derived three-dimensional state data as true value estimation data for estimating a three-dimensional state of the estimation target.

In other words, the three-dimensional state estimation device according to the first embodiment is configured to: set in advance in the statistical data setting means 1 the statistical data obtained by taking statistics of the correlation among the complexity indicator, the area fraction, and the three-dimensional state data, which serve as configuration data; derive, when the complexity indicator and the area fraction of the multicomponent material serving as the estimation target are input to the three-dimensional state estimation means 2, the three-dimensional state data corresponding to the input of the complexity indicator and the area fraction through collation with the statistical data; and directly output the derived three-dimensional state data as the true value estimation data on the estimation target. The three-dimensional state estimation device according to the first embodiment can be configured by a publicly known arithmetic processing device.

The complexity indicator quantitatively indicates diverse display states of the component of interest in the two-dimensional image data as the complexity of the image.

The type of the complexity indicator is not particularly limited, and an appropriate type of complexity indicator can be selected depending on the purpose. That is, the present invention has been made based on the knowledge that non-uniform display states of the component of interest in the two-dimensional image data, namely, various display states thereof may be an indicator useful for estimating the three-dimensional state of the multicomponent material, and it suffices that the complexity indicator can quantify the various display states.

Various types of statistics used for analysis of a texture image can be given as an indicator appropriate for the complexity indicator, and examples thereof comprise a statistical feature calculated due to a difference in the image density value by a statistical technique using, for example, a fractal dimension value, a density co-occurrence matrix, and a density difference vector, or in this description, a statistical feature calculated due to a difference in the image density value when different image density values are given to the component of interest and the component of non-interest.

Specific details of estimation processing using the complexity indicator are described later by separately taking a plurality of examples.

The area fraction is an area fraction of the two-dimensional image data occupied by the component of interest.

The three-dimensional state data is data on a content percentage of the component of interest in the multicomponent material at a time when the complexity indicator and the area fraction are determined.

Specifically, the three-dimensional state estimation device according to the first embodiment sets statistical data by the statistical data setting means 1 by adding the three-dimensional state data itself to the configuration data as the three-dimensional estimation data, and thus the true value estimation data is directly output by simply inputting the complexity indicator and the area fraction of the estimation target to the three-dimensional state estimation means 2 for collation with the statistical data.

The type of the three-dimensional state data is not particularly limited, and is preferably data useful for grasping the internal structure of the multicomponent material. The three-dimensional state data is preferably data such as the degree of liberation or the degree of locking, which is considered to be an indicator useful for evaluating the multicomponent material.

The degree of liberation indicates any one of the area fraction, volume fraction, mass fraction, and count fraction of the liberated particle in the group of particles, and the degree of locking indicates any one of the area fraction, volume fraction, mass fraction, and count fraction in the group of locked particles in which any one of the area fraction, volume fraction, and mass fraction of the component of interest in one particle is a fixed fraction.

As a method of setting the statistical data, as illustrated in FIG. 3, there is given a method of creating and registering, by the statistical data setting means 1, the statistical data by taking statistics of the correlation among the complexity indicator, the area fraction, and the three-dimensional state data serving as the three-dimensional estimation data based on input of those pieces of configuration data.

Although not shown, publicly known arithmetic processing means other than the three-dimensional state estimation device may create the statistical data by taking statistics of the correlation among the complexity indicator, the area fraction, and the three-dimensional state data, and input the created statistical data to the statistical data setting means 1 for registration.

The type of the method of creating the statistical data is not particularly limited, and may be any one of an empirical method or a non-empirical method.

For example, the empirical method involves: obtaining the two-dimensional image data on the multicomponent material to obtain the complexity indicator and the area fraction; empirically obtaining the three-dimensional state data by, for example, a method of continuously obtaining information on a plurality of cross-sections of the multicomponent material at minute intervals by repeating microscope observation and polishing of the multicomponent material after acquisition of the two-dimensional image data; and taking statistics of the correlation among the obtained complexity indicator, the area fraction, and the three-dimensional state data. The multicomponent material used in the empirical method is a sample for measurement of the multicomponent material of a target to be measured, and may be freely selected. For example, when the multicomponent material of the target to be measured is an ore, and the same type of ore is selected as a sample, a more accurate estimation result is likely to be easily obtained. Thus, the same type of multicomponent material as the multicomponent material of the target to be measured is preferably selected. Further, a plurality of types of statistical data created by the empirical method may be set so as to be selectable depending on the type of the multicomponent material of the target to be measured.

Further, for example, the non-empirical method involves: obtaining the complexity indicator and the area fraction based on the two-dimensional image data on the multicomponent material to which, for example, the three-dimensional state data, the number, size, and shape of constituent particles, and the distribution state of the component of interest in the constituent particles are virtually set in advance; and, for example, taking statistics of the correlation among the obtained complexity indicator, the area fraction, and the three-dimensional state data. Various kinds of data set in the non-empirical method are sample data for measurement of the multicomponent material of the target to be measured, and may be freely selected. For example, when the multicomponent material of the target to be measured is an ore and data is set in consideration of the same type of ore, a more accurate estimation result is likely to be easily obtained. Thus, the data is preferably set in consideration of the same type of multicomponent material as the multicomponent material of the target to be measured. A plurality of types of statistical data created by the non-empirical method may be set so as to be selectable depending on the type of the multicomponent material of the target to be measured.

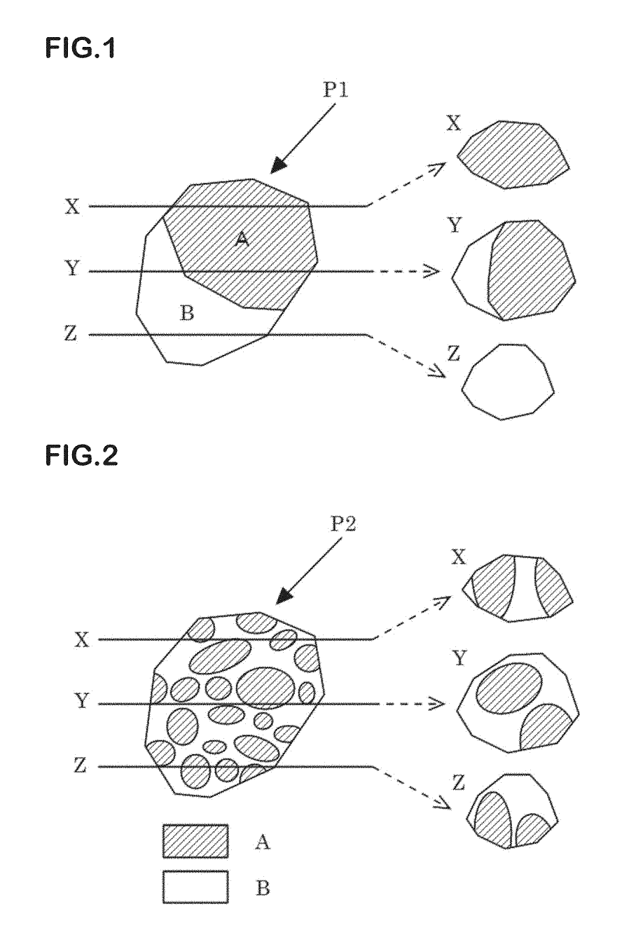

In any of the empirical method and non-empirical method, the three-dimensional state data is preferably obtained in such a manner as to correct the two-dimensional state data to the three-dimensional state data in accordance with a principle of occurrence of the stereological bias (refer to FIG. 1). Further, when the plurality of types of statistical data are set, those pieces of data may be created by any of the empirical method and non-empirical method, or may be a combination of a piece of data created by the empirical method or a piece of data created by the non-empirical method.

Regarding the complexity indicator and the area fraction serving as the data on the target to be measured, which are input to the three-dimensional state estimation means 2, publicly known image analysis means (not shown) may be provided in the three-dimensional state estimation device, and the image analysis means may, for example, obtain and analyze the two-dimensional image data serving as the data on the target to be measured to obtain the complexity indicator and the area fraction, or the complexity indicator and the area fraction may be obtained from the image analysis means outside the three-dimensional state estimation device.

In the following, a more specific description is given of a flow of estimation processing for each of a case in which the complexity indicator is set as a statistical feature calculated due to a difference in the image density value when different image density values are given to the component of interest and the component of non-interest by a statistical method that uses the fractal dimension value and the density co-occurrence matrix, and a case in which the complexity indicator is set as a statistical feature calculated due to a difference in the image density value when different image density values are given to the component of interest and the component of non-interest by a statistical method that uses the density difference vector.

First, a description is given of the flow of the estimation processing in a case where the fractal dimension value is used.

In this description, the fractal dimension value is denoted by δ, and is calculated in accordance with the following Equation (1).

$$\begin{matrix}[ Numerical Equation 2 ] & \\ δ = 2 - \frac{log A ( r ) - C}{log r} & ( 1 )\end{matrix}$$

In the said Equation (1), r indicates a length of one side of a defined square region, which is defined by equally dividing a square region with a length of one side being R in the two-dimensional image data into N² blocks by an integer N, A(r) indicates, when respective vertices of a square in the defined square region are denoted by A, B, C, and D, plane coordinates X and Y are set in the same plane as a plane of the respective vertices A, B, C, and D, and respective points set depending on image strengths at the respective vertices A, B, C, and D in the two-dimensional image data as a height Z in a direction orthogonal to the plane forming the plane coordinates X and Y are denoted by set points A′, B′, C′, and D′, a sum of areas of two triangles, namely, one triangle having the set points A′, B′, and D′ as vertices, and the other triangle having the set points B′, C′, and D′ as vertices, which are calculated for all the defined square regions in the square region, and C indicates log A(1).

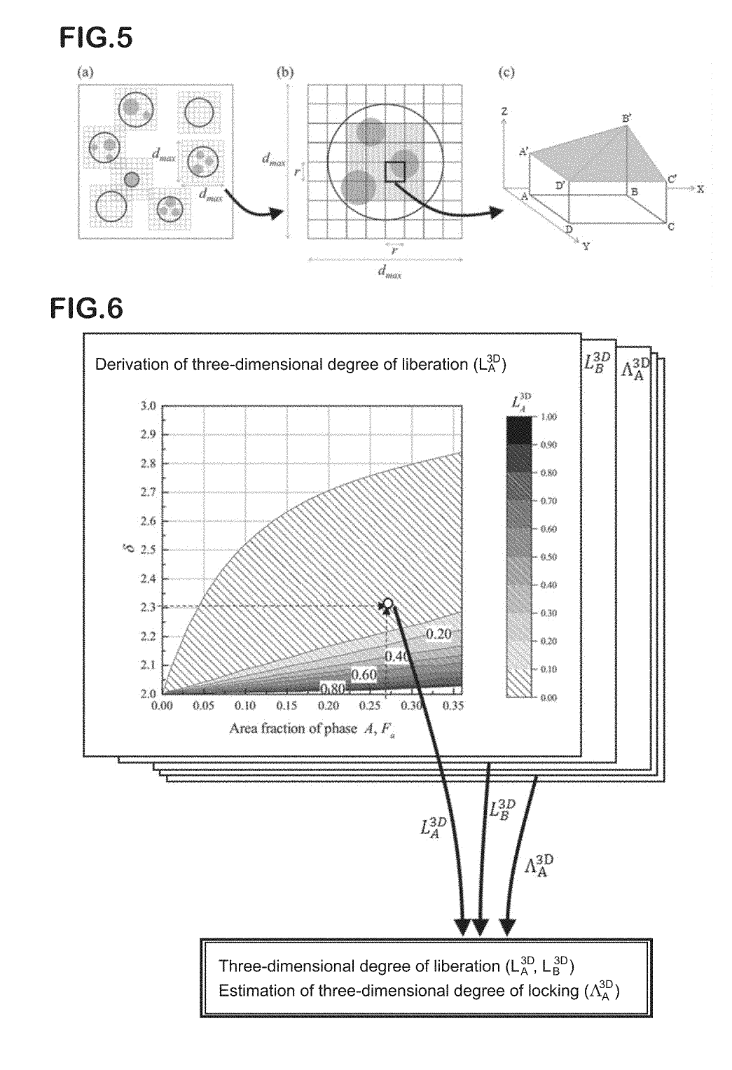

Specifically, first, the cross-section of one particle is focused in the two-dimensional image data illustrated in FIG. 5(a), and a square region with the length of one side being R, which is substantially equal to the maximum diameter (dₘₐₓ) of the particle illustrated in FIG. 5(b), is set. FIG. 5 are explanatory diagrams for illustrating a process of calculating the fractal dimensional value δ.

Next, this square region is equally divided into N² blocks by a freely selected integer N (N=8 in the example illustrated in FIG. 5(b)), and a defined square region with the length of one side being r is set.

Next, respective vertices of a square in the defined square region are denoted by A, B, C, and D, the plane coordinates X and Y are set in the same plane as that of the respective vertices A, B, C, and D, and respective points set depending on image strengths (each image density value in the example of FIG. 5) at the respective vertices A, B, C, and D in the two-dimensional image data as the height Z in a direction orthogonal to the plane forming the plane coordinates X and Y are denoted by the set points A′, B′, C′, and D′. The set points A′, B′, C′, and D′ correspond to the vertices A, B, C, and D as illustrated in FIG. 5(c) in alphabets, respectively.

Next, areas of two triangles, namely, one triangle having the set points A′, B′, and D′ as its vertices and the other triangle having the set points B′, C′, and D′ as its vertices are calculated. This calculation is performed for all the defined square regions in the square region, and the sum thereof is set as A(r).

Next, r and A(r) are substituted into the said Equation (1) to obtain the fractal dimensional value δ.

In order to reduce a probability error, it is preferred that the processing of calculating the above-mentioned fractal dimensional value δ be performed a plurality of times by, for example, changing the value of N, and the plurality of calculated fractal dimensional values δ be used to approximate one definite fractal dimensional value δ by a least-square method.

Further, a fractal dimension value representing the entire two-dimensional image data can be calculated by dividing a sum of products of the fractal dimensional value δ calculated for the cross-section of each particle with the above-mentioned method and the cross-sectional area of each particle, which are calculated for the cross-sections of particles in the entire two-dimensional image data, by a sum of cross-sectional areas of all the particles in the cross-section of the sample of the multicomponent material in the two-dimensional image data.

The calculation method described in the following Reference Document 1 can be referred to for the processing of calculating the fractal dimension value. Reference Document 1: H. Kaneko, Fractal Feature and Texture Analysis, IEICE Trans. Inf. Syst. (Japanese Ed. J70-D (1987) 964-972.

Next, the area fraction Fa of an area (part with high image density) of the component of interest is calculated from the two-dimensional image data illustrated in FIG. 5(a).

The processing of calculating the fractal dimensional value δ and the area fraction Fa described above is performed for each of the plurality of pieces of two-dimensional image data of the multicomponent material having various values of the three-dimensional state data to take statistics of the correlation among the fractal dimensional value δ, the area fraction Fa, and the value of three-dimensional state data, and the statistical data representing, as a contour line diagram, the correlation among the fractal dimensional value δ, the area fraction Fa, and the value of three-dimensional state data, for example, is created as illustrated in FIG. 6, and registered in the statistical data setting means 1. That is, the statistical data is set in the statistical data setting means 1.

FIG. 6 is a diagram for illustrating, as a contour line diagram, statistical data obtained by taking statistics of the correlation among the fractal dimensional value δ, the area fraction Fa, and the value of three-dimensional state data. Further, “L_{A}^{3D}” in FIG. 6 indicates the degree of liberation of the component A, “L_{B}^{3D}” indicates the degree of liberation of the component B, and “Λ_{A}^{3D}” indicates the degree of locking focused on the component A as the three-dimensional state data.

Next, with processing similar to the processing of setting the statistical data, the fractal dimensional value δ and the area fraction Fa are obtained for the multicomponent material serving as the estimation target.

The obtained fractal dimensional value δ and area fraction Fa are input to the three-dimensional state estimation means 2 for collation with the statistical data, and the three-dimensional state data corresponding to those input values is obtained to be directly output as the true value estimation data (refer to FIG. 6).

Through the estimation processing described above, the three-dimensional state estimation device according to the first embodiment can estimate the true value of the three-dimensional state data on the multicomponent material based on the two-dimensional image data.

Next, a description is given of the estimation processing for a case of using a statistical feature calculated due to a difference in the image density value when different image density values are given to the component of interest and the component of non-interest by a statistical method that uses the density co-occurrence matrix. This estimation processing is similar to that of using the fractal dimensional value δ except that the density co-occurrence matrix is used as the statistical feature instead of the fractal dimensional value δ. Thus, in the following, a description is given of a method of calculating the statistical feature and the statistical data obtained from this statistical feature.

The density co-occurrence matrix P(i,j:d,θ) is a matrix indicating, when an entire or partial region of two-dimensional image data represented by two or more tones of the density level is observed with XY plane coordinates, the frequency in that region of a pair of a pixel 1 with a pixel density value of i and a pixel 2 with a pixel density value of j, which are any two pixels in that region, where d represents a coordinate distance between the pixel 1 and the pixel 2 and θ represents an angle formed by a straight line connecting those two pixels and the X axis.

The total density co-occurrence matrix P(i,j) represented by the following Equation (2) is derived from the density co-occurrence matrix P(i,j:d,θ). The total density co-occurrence matrix P(i,j) is a matrix obtained by adding the density co-occurrence matrices P for all θ with respect to the density co-occurrence matrix P(i,j:d,θ) having the same d and different θ.

$$\begin{matrix}[ Numerical Equation 3 ] & \\ P ( i , j ) = {\sum }_{θ} P ( i , j ; d , θ ) & ( 2 )\end{matrix}$$

Further, a differential density co-occurrence matrix P^{r}(i,j) represented by the following Equation (3) is derived from the density co-occurrence matrix P(i,j:d,θ). This differential density co-occurrence matrix P^{r}(i,j) is a matrix obtained by taking a difference between the maximum value and the minimum value for each element of the density co-occurrence matrix P(i,j:d,θ) with the same d and different θ.

[Numerical Equation 4]

P^{r}(i,j)=max(P(i,j;dθ)−min(P(i,j;d,θ)) (3)

The total density co-occurrence matrix P(i,j) is normalized by a sum of elements, and the normalized total density co-occurrence matrix p(i,j) represented by the following Equation (4) is derived.

$$\begin{matrix}[ Numerical Equation 5 ] & \\ p ( i , j ) = \frac{P ( i , j )}{{\sum }_{i = 1}^{g} {\sum }_{j = 1}^{g} P ( i , j )} & ( 4 )\end{matrix}$$

The differential density co-occurrence matrix P^{r}(i,j) is normalized by a sum of elements, and the differential density co-occurrence matrix p^{r}(i,j) represented by the following Equation (5) is derived.

$$\begin{matrix}[ Numerical Equation 6 ] & \\ {p}^{r} ( i , j ) = \frac{{P}^{r} ( i , j )}{{\sum }_{i = 1}^{g} {\sum }_{j = 1}^{g} {P}^{r} ( i , j )} & ( 5 )\end{matrix}$$

In this manner, the normalized total density co-occurrence matrix p(i,j) obtained by normalizing a sum of elements of the density co-occurrence matrices P(i,j;dθ) and the normalized differential density co-occurrence matrix p^{r}(i,j) obtained by normalizing a difference between the maximum value and minimum value of elements of the density co-occurrence matrix P(i,j;dθ) are derived.

The normalized total density co-occurrence matrix p(i,j) is used to calculate the statistical feature. In this description, the following 14 types of statistical features (f₁ to f₁₄) proposed by Haralick are exemplified as a method of analyzing the texture of the two-dimensional image. Similar statistical features (f₁^{r} to f₁₄^{r}) are obtained by using the normalized differential density co-occurrence matrix p^{r}(i,j) instead of the normalized total density co-occurrence matrix p(i,j).

$$\begin{matrix}[ Numerical Equation 7 ] & \\ {f}_{1} \text{:} Angular Second Moment & \\ {f}_{1} = {\sum }_{i} {\sum }_{j} {\{ p ( i , j ) \}}^{2} & ( 6 ) \\ {f}_{2} \text{:} Contrast & \\ {f}_{2} = {\sum }_{n = 0}^{g - 1} {n}^{2} \{ {\sum }_{i} {\sum }_{j} p ( i , j ) \} & ( 7 ) \\ {f}_{3} \text{:} Correlation & \\ {f}_{3} = \frac{{\sum }_{i} {\sum }_{j} ( ij ) p ( i , j ) - {μ}_{x} {μ}_{y}}{{σ}_{x} {σ}_{y}} & ( 8 ) \\ {f}_{4} \text{:} Sum of Squares & \\ {f}_{4} = {\sum }_{i} {\sum }_{j} {( i - μ )}^{2} p ( i , j ) & ( 9 ) \\ with the proviso that & \\ μ = \frac{{\sum }_{i = 1}^{g} {p}_{x} ( i ) + {\sum }_{j = 1}^{g} {p}_{y} ( j )}{2} & ( 10 ) \\ {p}_{x} ( i ) = {\sum }_{j = 1}^{g} p ( i , j ) & ( 11 ) \\ {p}_{y} ( j ) = {\sum }_{i = 1}^{g} p ( i , j & ( 12 ) \\ [ Numerical Equation 8 ] & \\ {f}_{5} \text{:} Inverse Difference Moment & \\ {f}_{5} = {\sum }_{i} {\sum }_{j} \frac{p ( i , j )}{1 + {( i - j )}^{2}} & ( 13 ) \\ {f}_{6} \text{:} Sum Average & \\ {f}_{6} = {\sum }_{i = 2}^{2 g} {ip}_{x + y} ( i ) & ( 14 ) \\ with the proviso that & \\ {p}_{x + y} ( k ) = {\sum }_{i} {\sum }_{j} p ( i , j ) , k = i + j & ( 15 ) \\ {f}_{7} \text{:} Sum Variance & \\ {f}_{7} = {\sum }_{i = 2}^{2 g} {( i - {f}_{8} )}^{2} {p}_{x + y} ( i ) & ( 16 ) \\ {f}_{8} \text{:} Sum Entropy & \\ {f}_{8} = - {\sum }_{i = 2}^{2 g} {p}_{x + y} ( i ) ln \{ {p}_{x + y} ( i ) \} & ( 17 ) \\ {f}_{9} \text{:} Entropy & \\ {f}_{9} = - {\sum }_{i} {\sum }_{j} p ( i , j ) ln \{ p ( i , j ) \} & ( 18 )\end{matrix}$$

$$\begin{matrix}[ Numerical Equation 9 ] & \\ {f}_{10} \text{:} Difference Variance & \\ {f}_{10} = \frac{{\sum }_{k = 0}^{g - 1} {\{ {p}_{( x - y )} ( k ) - \sum \frac{{p}_{x - y} ( k )}{g} \}}^{2}}{g - 1} & ( 19 ) \\ with the proviso that & \\ {p}_{x - y} ( k ) = {\sum }_{i} {\sum }_{j} p ( i , j ) , k = \vert i - j \vert & ( 20 ) \\ {f}_{11} \text{:} Difference Entropy & \\ {f}_{11} = - {\sum }_{i = 0}^{2 g} {p}_{x - y} ( i ) ln \{ {p}_{x - y} ( i ) \} & ( 21 ) \\ {f}_{12 ‵} {f}_{13} \text{:} Information Measures of Correlation & \\ {f}_{12} = \frac{HXY - HXY 1}{max \{ HX , HY \}} & ( 22 ) \\ {f}_{13} = {( 1 - exp [ - 2.0 ( HXY 2 - HXY ) ] )}^{\frac{1}{2}} & ( 23 ) \\ with the proviso that & \\ HXY = - {\sum }_{i} {\sum }_{j} p ( i , j ) ln \{ p ( i , j ) \} & ( 24 ) \\ HXY 1 = - {\sum }_{i} {\sum }_{j} p ( i , j ) ln \{ {p}_{x} ( i ) {p}_{y} ( j ) \} & ( 25 ) \\ HXY 2 = - {\sum }_{i} {\sum }_{j} {p}_{x} ( i ) {p}_{y} ( f ) ln \{ {p}_{x} ( i ) {p}_{y} ( j ) \} & ( 26 ) \\ HX = - {\sum }_{i} {p}_{x} ( i ) ln \{ {p}_{x} ( i ) \} & ( 27 ) \\ HY = - {\sum }_{i} {p}_{y} ( i ) ln \{ {p}_{y} ( i ) \} & ( 28 ) \\ {f}_{14} \text{:} Maximal Correlation Coefficient & \\ {f}_{14} = {( Second largest eigenvalue of T )}^{\frac{1}{2}} \\ with the proviso that & ( 29 ) \\ T ( i , j ) = {\sum }_{k} \frac{p ( i , k ) p ( j , k )}{{p}_{x} ( i ) {p}_{y} ( k )} & ( 30 )\end{matrix}$$

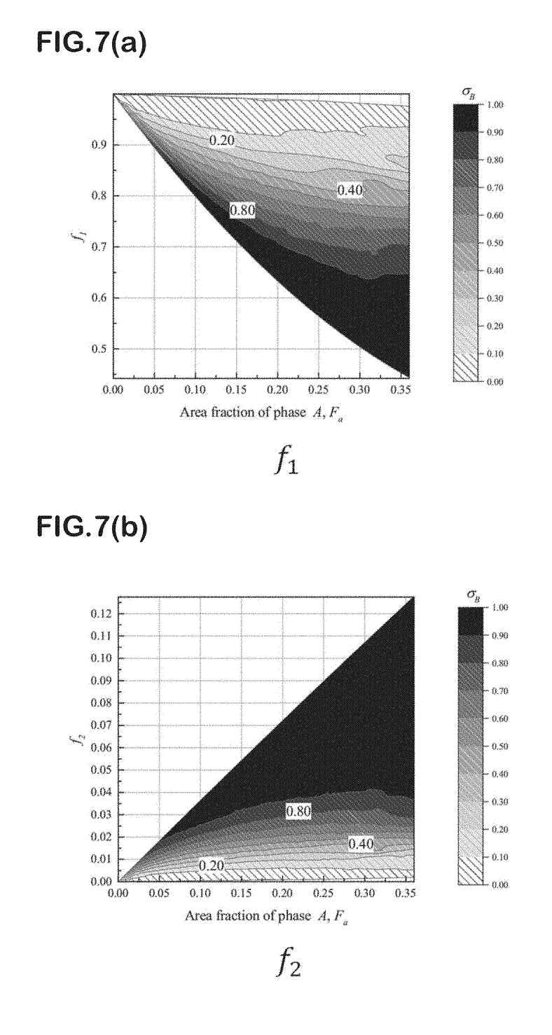

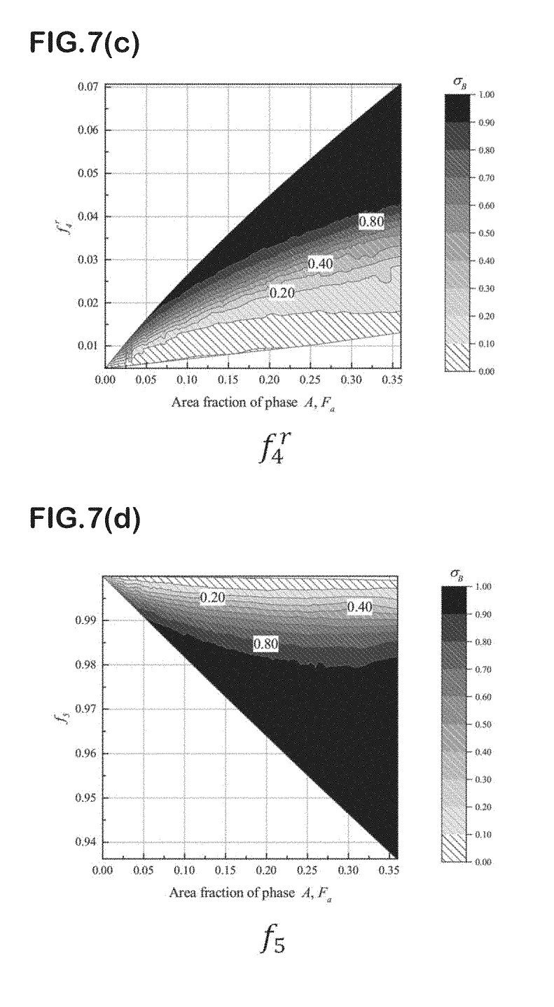

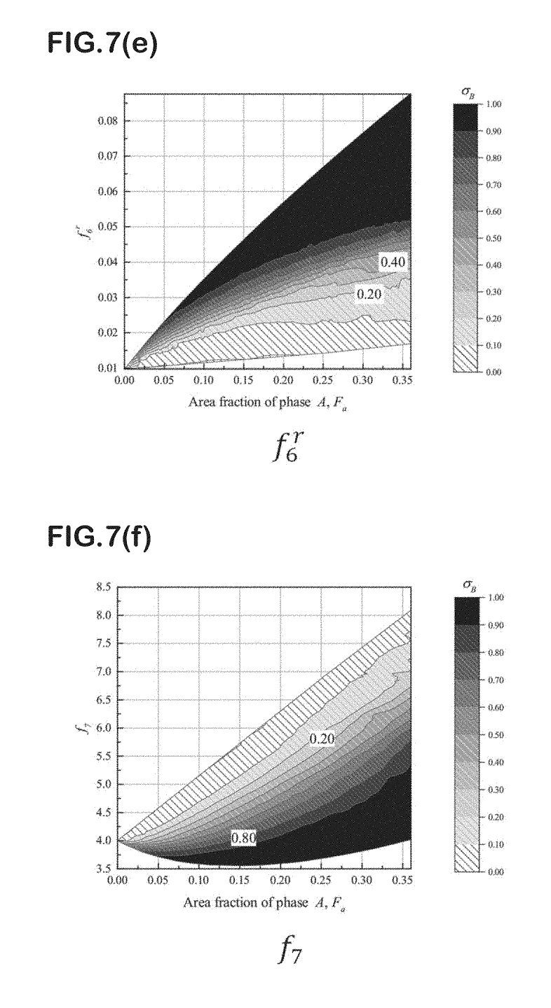

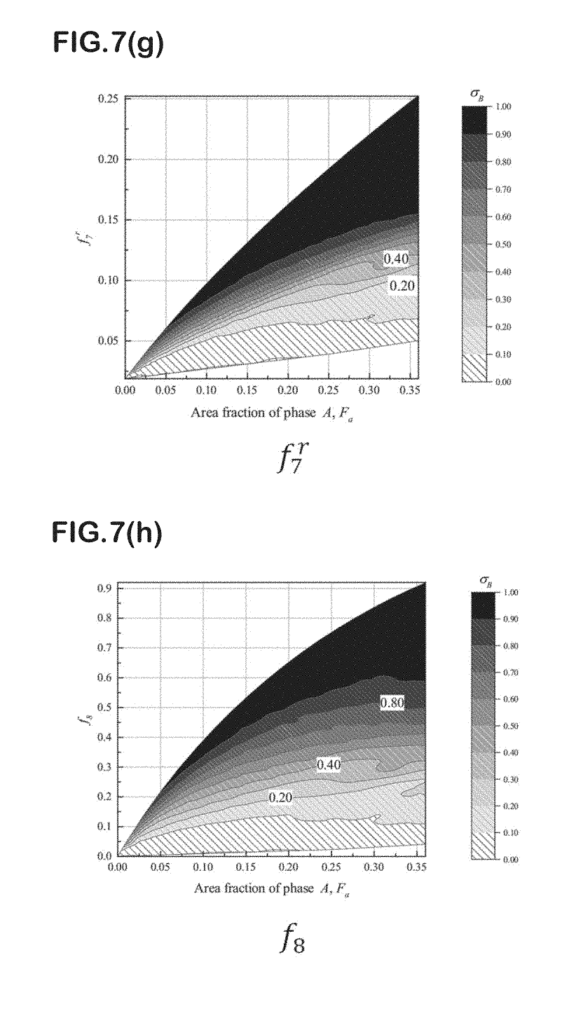

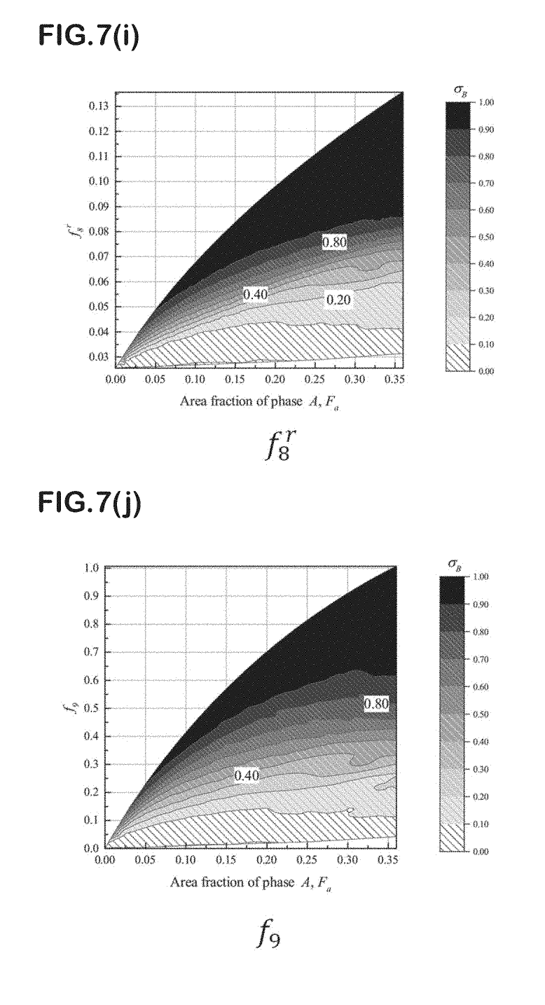

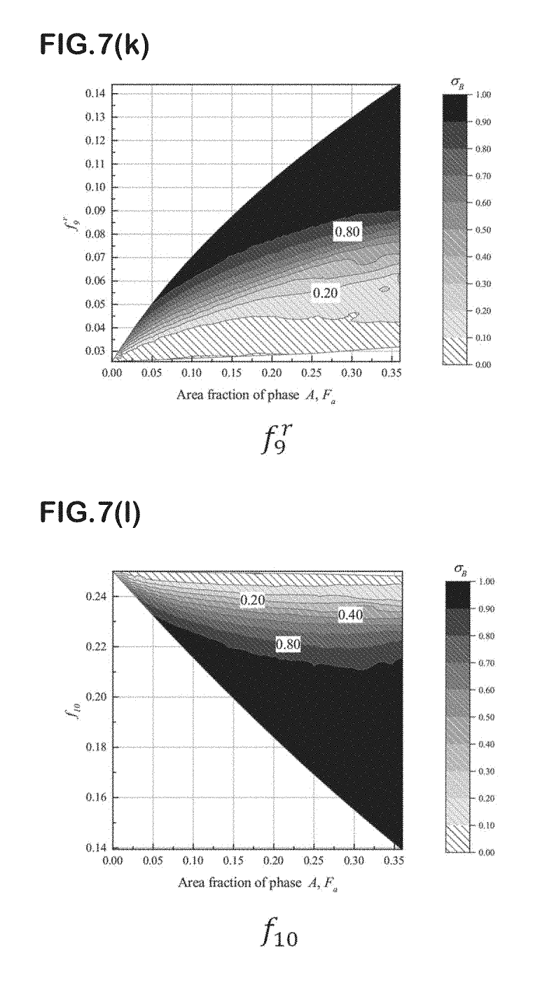

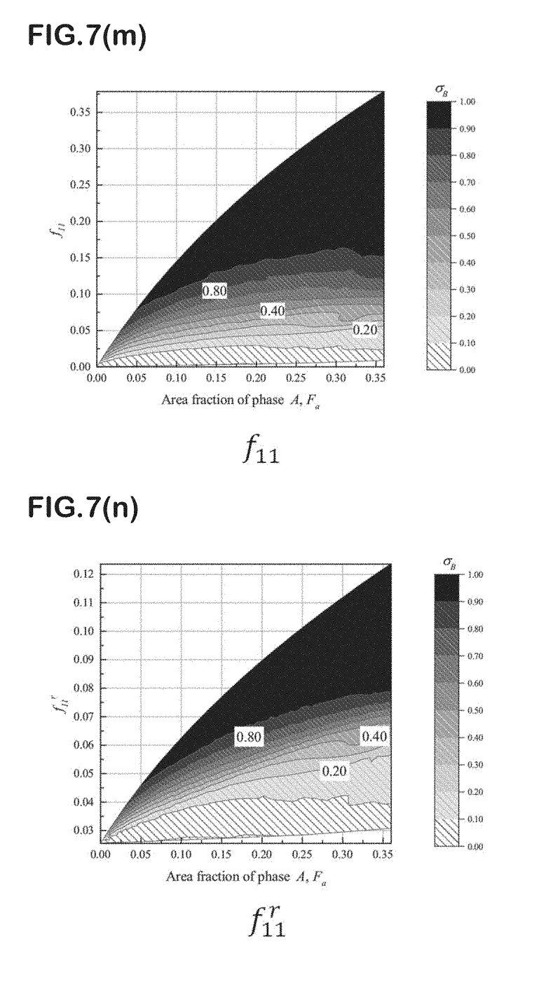

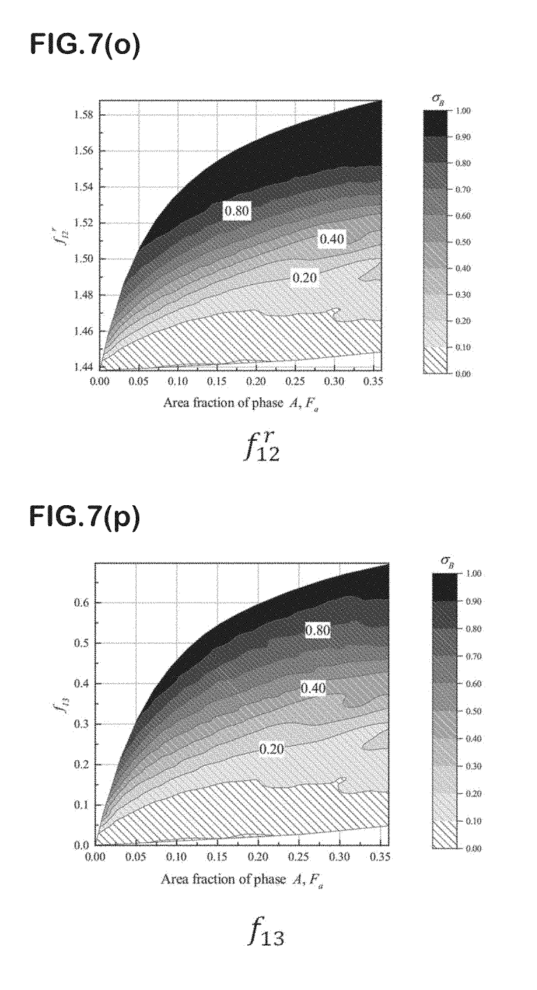

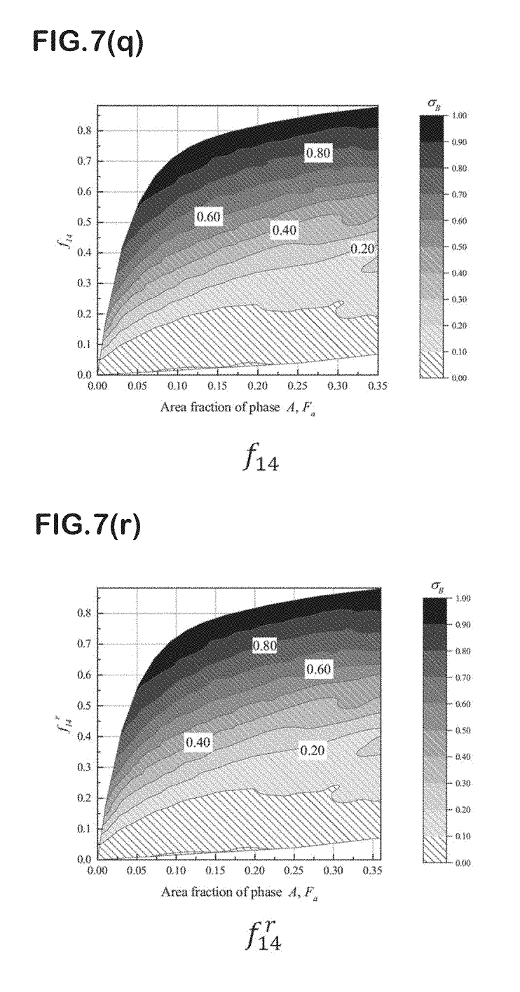

Examples of the statistical data obtained from the statistical features are shown in FIG. 7(a) to FIG. 7(r). FIG. 7(a) is a contour line diagram obtained by setting f₁ as the statistical feature, FIG. 7(b) is a contour line diagram obtained by setting f₂ as the statistical feature, FIG. 7(c) is a contour line diagram obtained by setting f₄^{r} as the statistical feature, FIG. 7(d) is a contour line diagram obtained by setting f₅ as the statistical feature, FIG. 7(e) is a contour line diagram obtained by setting f₆^{r} as the statistical feature, FIG. 7(f) is a contour line diagram obtained by setting f₇ as the statistical feature, FIG. 7(g) is a contour line diagram obtained by setting f₇^{r} as the statistical feature, FIG. 7(h) is a contour line diagram obtained by setting f₈ as the statistical feature, FIG. 7(i) is a contour line diagram obtained by setting f₈^{r} as the statistical feature, FIG. 7(j) is a contour line diagram obtained by setting f₉ as the statistical feature, FIG. 7(k) is a contour line diagram obtained by setting f₉^{r} as the statistical feature, FIG. 7(l) is a contour line diagram obtained by setting f₁₀ as the statistical feature, FIG. 7(m) is a contour line diagram obtained by setting f₁₁ as the statistical feature, FIG. 7(n) is a contour line diagram obtained by setting f₁₁^{r} as the statistical feature, FIG. 7(o) is a contour line diagram obtained by setting f₁₂^{r} as the statistical feature, FIG. 7(p) is a contour line diagram obtained by setting f₁₃ as the statistical feature, FIG. 7(q) is a contour line diagram obtained by setting f₁₄ as the statistical feature, and FIG. 7(r) is a contour line diagram obtained by setting f₁₄^{r} as the statistical feature.

In creation of the statistical data shown in FIG. 7(a) to FIG. 7(r), the number of tones of the two-dimensional image data is set to 2, the coordinates of the pixel 1 are to set to (x, y), the coordinates of the pixel 2 are set to 8 patterns, namely, (x+1, y), (x−1, y), (x+1, y+1), (x−1, y−1), (x, y+1), (x, y−1), (x−1, y+1), (x+1, y−1), and d is set to 1. Further, the angle θ formed by a straight line connecting the pixel 1 and the pixel 2 and the X axis is set to 4 patterns, namely, 0° for the coordinates (x+1, and (x−1, y) of the pixel 2, 45° for the coordinates (x+1, y+1) and (x−1, y−1) of the pixel 2, 90° for the coordinates (x, y+1) and (x, y−1) of the pixel 2, and 135° for the coordinates (x−1, y+1) and (x+1, y−1) of the pixel 2. Those four patterns of the density co-occurrence matrix P(i,j;dθ), namely,)P(i,j:1,0°), P(i,j:1,45°), P(i,j:1,90°), and)P(i,j:1,135°) are used to perform various kinds of calculations. The coordinate distance d between the pixel 1 and the pixel 2 is calculated to be a square root of (x−x2)²+(y−y2)² when the coordinates of the pixel 1 and the pixel 2 are represented by (x, y) and (x2, y2), respectively, and is rounded off to an integer when d is a mixed decimal. Thus, when the angle θ represents 0° or 90°, d=1 is satisfied, whereas d=√/2 is satisfied when the angle θ represents 45° or 135°. In the latter case, d is rounded off to 1, and thus d=1 is satisfied in both cases.

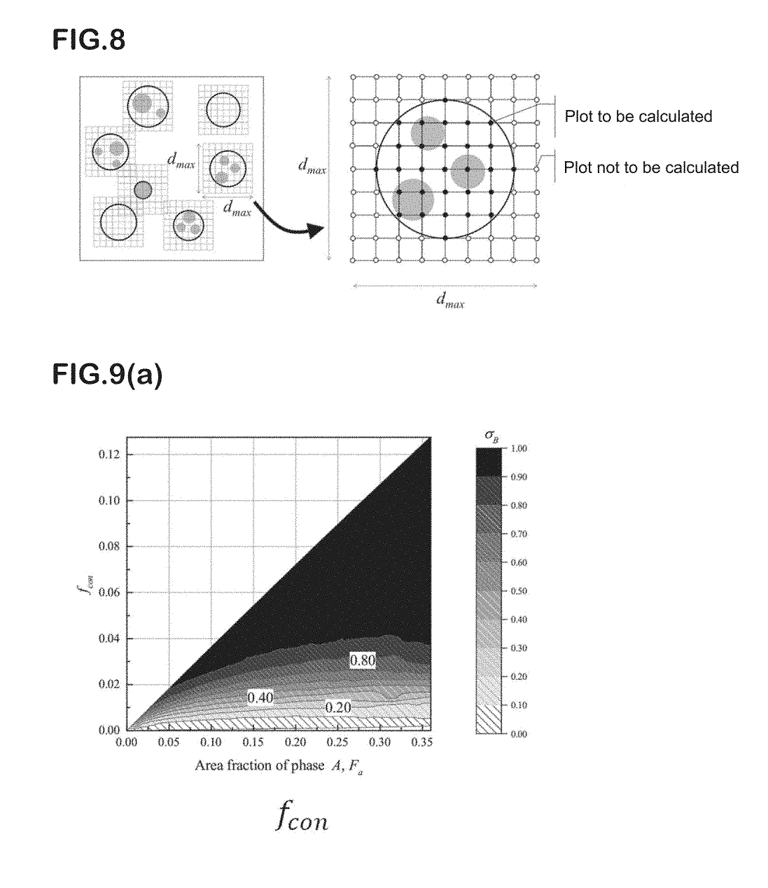

Further, in calculation, as illustrated in FIG. 8, a square region with the length of one side being dₘₐₓ and having the same center as that of each particle cross-section is set, the square region is equally divided into (N−1)² blocks to create N² plots, and the calculation is performed for a plot within the particle cross-section among the created N² plots. In this case, N is set to 50. FIG. 8 is an explanatory diagram for illustrating a calculation region.

Next, a description is given of the estimation processing for a case of using a statistical feature calculated due to a difference in the image density value when different image density values are given to the component of interest and the component of non-interest by a statistical method that uses the density difference vector. This estimation processing is similar to that of using the fractal dimensional value δ except that the density co-occurrence matrix is used as the statistical feature instead of the fractal dimensional value δ. Thus, in the following, a description is given of a method of calculating the statistical feature and the statistical data obtained from this statistical feature.

The density difference vector Q(i:d,θ) is a vector indicating, when an entire or partial region of two-dimensional image data represented by two or more tones of the density level is observed with XY plane coordinates, the frequency in that region of a pair of a pixel 1 and a pixel 2, which are any two pixels in that region, with a difference between a pixel density value of the pixel 1 and pixel density value of the pixel 2 being i, where d represents a coordinate distance between the pixel 1 and the pixel 2 and θ represents an angle formed by a straight line connecting those two pixels and the X axis.

The total density difference vector Q(i) represented by the following Equation (31) is derived from the density difference vector Q(i:d,θ). The total density difference vector Q(i) is a vector obtained by adding the density difference vectors Q for all θ with respect to the density difference vectors Q(I:d,θ) having the same d and different θ.

$$\begin{matrix}[ Numerical Equation 10 ] & \\ Q ( i ) = {\sum }_{θ} Q ( i ; d , θ ) & ( 31 )\end{matrix}$$

Further, a differential density difference vector Q^{r}(i) represented by the following Equation (32) is derived from the density difference vector Q(i:d,θ). This differential density difference vector Q^{r}(i) is a vector obtained by taking a difference between the maximum value and the minimum value for each element of the density difference vector Q(i:d,θ) with the same d and different θ.

[Numerical Equation 11]

Q^{r}(i)=max(Q(i;d,θ)−min(Q(i;d,θ) (32)

Further, the total density difference vector Q(i) is normalized by a sum of elements, and the normalized total density difference vector q(i) represented by the following Equation (33) is derived.

$$\begin{matrix}[ Numerical Equation 12 ] & \\ q ( i ) = \frac{Q ( i )}{{\sum }_{i = 1}^{g} Q ( i )} & ( 33 )\end{matrix}$$

The differential density difference vector Q^{r}(i) is normalized by a sum of elements, and the normalized differential density difference vector q^{r}(i) represented by the following Equation (34) is derived.

$$\begin{matrix}[ Numerical Equation 13 ] & \\ {q}^{r} ( i ) = \frac{{Q}^{r} ( i )}{{\sum }_{i = 1}^{g} {Q}^{r} ( i )} & ( 34 )\end{matrix}$$

In this manner, the normalized total density difference vector q(i) obtained by normalizing a sum of elements of the density difference vector Q(i:d,θ) and the normalized differential density difference vector q^{r}(i) obtained by normalizing a difference between the maximum value and minimum value of elements of the density difference vector Q(i:d,θ) are derived.

The normalized total density difference vector q(i) is used to calculate the statistical features. In this description, the following four types of statistical features (f_{con}, fₐₛₘ, fₑₙₜ, fₘₑₐₙ) used for the method of analyzing the texture of the two-dimensional image are exemplified. Similar statistical features (f_{con}^{r}, fₐₛₘ^{r}, fₑₙₜ^{r}, fₘₑₐₙ^{r}) are obtained by using the normalized difference density difference vector q^{r}(i) instead of the normalized total density difference vector q(i).

$$\begin{matrix}[ Numerical Equation 14 ] & \\ {f}_{con} \text{:} Contrast \\ {f}_{con} = {\sum }_{i = 0}^{g - 1} {i}^{2} q ( i ) & ( 35 ) \\ {f}_{asm} \text{:} Angular Second Moment \\ {f}_{asm} = {\sum }_{i = 0}^{g - 1} {\{ iq ( i ) \}}^{2} & ( 36 ) \\ {f}_{ent} \text{:} Entropy \\ {f}_{ent} = - {\sum }_{i = 0}^{g - 1} q ( i ) ln \{ q ( i ) \} & ( 37 ) \\ {f}_{mean} \text{:} Mean \\ {f}_{mean} = {\sum }_{i = 0}^{g - 1} iq ( i ) & ( 38 )\end{matrix}$$

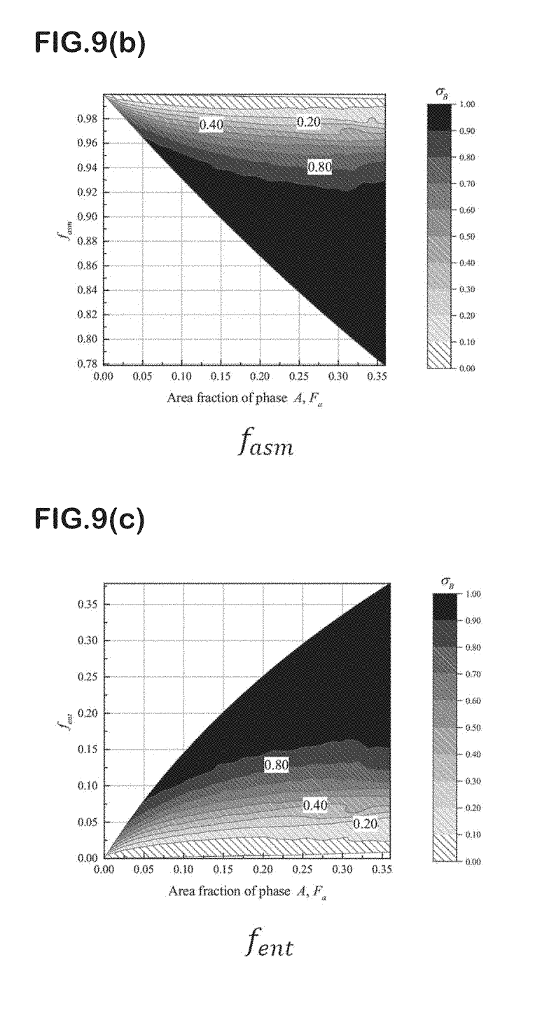

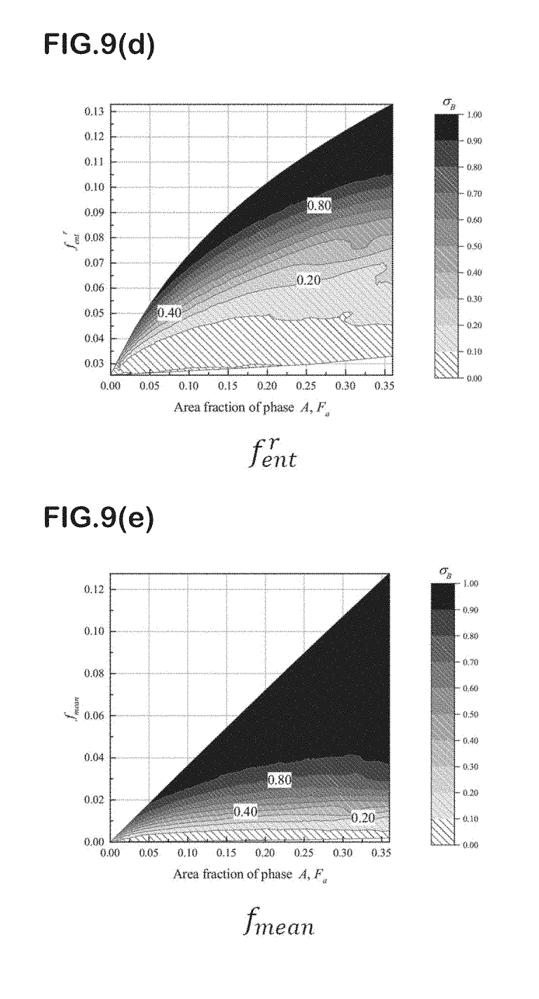

Examples of the statistical data obtained from the statistical features are shown in FIG. 9(a) to FIG. 9(e). FIG. 9(a) is a contour line diagram obtained by setting f_{con} as the statistical feature, FIG. 9(b) is a contour line diagram obtained by setting fₐₛₘ as the statistical feature, FIG. 9(c) is a contour line diagram obtained by setting fₑₙₜ^{r} as the statistical feature, FIG. 9(d) is a contour line diagram obtained by setting fₑₙₜ^{r} as the statistical feature, and FIG. 9(e) is a contour line diagram obtained by setting fₘₑₐₙ as the statistical feature.

Further, in creation of the statistical data shown in FIG. 9(a) to FIG. 9(e), as in the processing of the density co-occurrence matrix P(i,j:d,θ), the number of tones of the two-dimensional image data is set to 2, the coordinates of the pixel 1 are to set to (x, y), the coordinates of the pixel 2 are set to 4 patterns, namely, (x+1, y), (x+1, y+1), (x, y+1), (x−1, y+1), and d is set to 1. Further, the angle θ formed by a straight line connecting the pixel 1 and the pixel 2 and the X axis is set to 4 patterns, namely, 0° for the coordinates (x+1, y) of the pixel 2, 45° for the coordinates (x+1, y+1) of the pixel 2, 90° for the coordinates (x, y+1) of the pixel 2, and 135° for the coordinates (x−1, y+1) of the pixel 2. Those four patterns of the density difference vector Q(i:d,θ), namely,)Q(i:1,0°), Q(i:1,45°), Q(i:1,90°), and Q(i:1,135° are used to perform various kinds of calculations.

Further, in calculation, as illustrated in FIG. 8, a square region with the length of one side being dₘₐₓ and having the same center as that of each particle cross-section is set, the square region is equally divided into (N−1)² blocks to create N² plots, and the calculation is performed for a plot within the particle cross-section among the created N² plots. In this case, N is set to 50.

### Second Embodiment

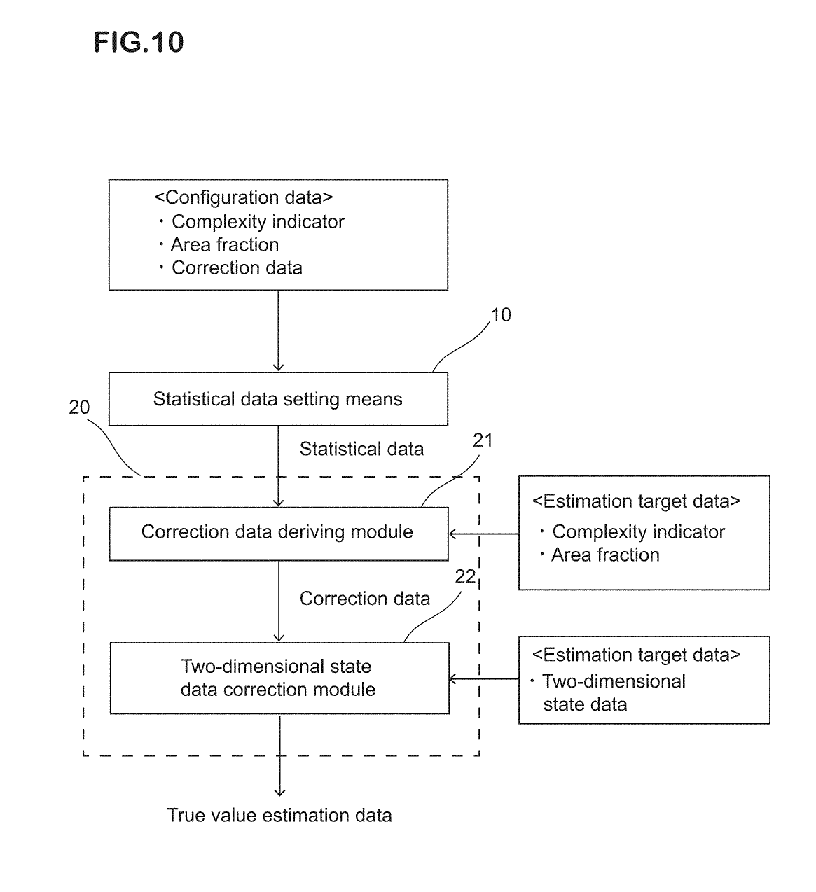

Next, a description is given of the second embodiment according to a three-dimensional state estimation device of the present invention. FIG. 10 is a block diagram for illustrating a configuration of the three-dimensional state estimation device and a flow of estimation processing in the second embodiment.

As illustrated in FIG. 10, the three-dimensional state estimation device according to the second embodiment comprises statistical data setting means 10 and three-dimensional state estimation means 20.

The statistical data setting means 10 can set statistical data obtained by taking statistics of a correlation among the complexity indicator, the area fraction, and correction data serving as the three-dimensional estimation data.

The correction data is data for correcting, to the three-dimensional state data, two-dimensional state data on the content percentage of the component of interest in the cross-section or surface of the multicomponent material, and is obtained based on the empirical or non-empirical method similarly to the three-dimensional state data described in the first embodiment.

The three-dimensional state estimation means 20 further comprises: a correction data deriving module 21 configured to collate input of the complexity indicator and the area fraction of the multicomponent material serving as the estimation target with the statistical data set in the statistical data setting means 10, to thereby derive the correction data corresponding to the input of the complexity indicator and the area fraction; and a two-dimensional state data correction module 22 configured to correct the input two-dimensional state data on the multicomponent material serving as the estimation target through use of the correction data derived by the correction data deriving module 21, to thereby derive the three-dimensional state data, and capable of outputting the derived three-dimensional state data as true value estimation data for estimating the three-dimensional state of the estimation target.

The three-dimensional state estimation device according to the second embodiment first sets in advance in the statistical data setting means 10 the statistical data obtained by taking statistics of the correlation among the complexity indicator, the area fraction, and the correction data, which serve as configuration data. Next, when the complexity indicator and the area fraction of the multicomponent material serving as the estimation target are input to the correction data deriving module 21 of the three-dimensional state estimation means 20, the three-dimensional state estimation device collates the complexity indicator and the area fraction with the statistical data to derive the correction data corresponding to the input of the complexity indicator and the area fraction. Next, the two-dimensional state data input to the two-dimensional state data correction module 22 of the three-dimensional state estimation means 20 is corrected with the correction data, and this two-dimensional state data is output as the true value estimation data on the estimation target. The three-dimensional state estimation device according to the second embodiment can be configured by a publicly known arithmetic processing device.

The three-dimensional state estimation device according to the first embodiment collates the input complexity indicator and area fraction of the multicomponent material serving as the estimation target with the complexity indicator and area fraction in the statistical data to directly output the three-dimensional state data corresponding to the complexity indicator and the area fraction as the true value estimation data, whereas the three-dimensional state estimation device according to the second embodiment collates the input complexity indicator and area fraction of the multicomponent material serving as the estimation target with the complexity indicator and area fraction in the statistical data, temporarily derives the correction data corresponding to the complexity indicator and the area fraction, corrects the separately input two-dimensional state data with the correction data, and outputs the two-dimensional state data as the true value estimation data on the estimation target.

Specifically, the three-dimensional state estimation device according to the second embodiment clarifies the stereological bias of the two-dimensional image data, which has been described through use of the model illustrated in FIG. 1, corrects the two-dimensional state data with the correction data so that the stereological bias hindering estimation of the three-dimensional state is directly removed, and obtains the true value estimation data on the estimation target indirectly from the statistical data. The stereological bias indicates a probability of the apparent degree of liberation observed from the two-dimensional image data causing overestimation of the degree of liberation in the three-dimensional state (refer to FIG. 1).

Then, the three-dimensional state estimation device according to the second embodiment obtains the two-dimensional state data in addition to the statistical data, and the complexity indicator and area fraction of the estimation target as input data. Thus, characteristics of the estimation target are estimated from a larger number of pieces of input data, and the true value estimation data having a higher estimation accuracy than the three-dimensional state estimation device according to the first embodiment can be obtained.

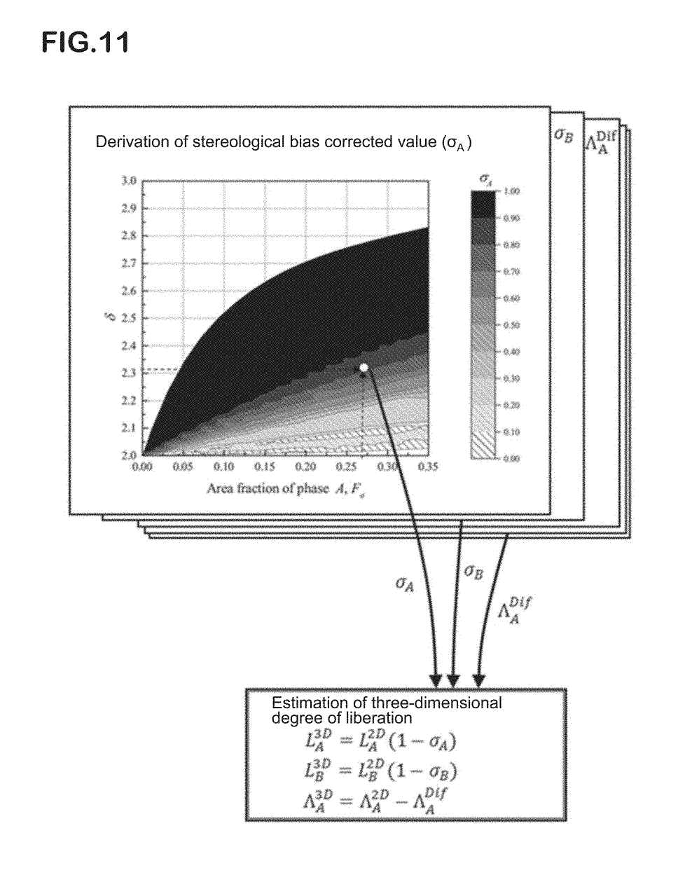

The three-dimensional state estimation device according to the second embodiment is similar to the three-dimensional state estimation device according to the first embodiment except that the true value estimation data of the estimation target is obtained from the correction data. For example, the three-dimensional state estimation device according to the second embodiment sets the statistical data illustrated in FIG. 11, and the true value estimation data on the estimation target is obtained based on the statistical data obtained by taking statistics of the correlation among the correction data (stereological bias corrected value in this description) instead of the three-dimensional state data, the complexity indicator, and the area fraction.

FIG. 11 is a diagram for illustrating, as a contour line diagram, the statistical data obtained by taking statistics of the correlation among the fractal dimensional value δ, the area fraction Fa, and the value of the stereological bias corrected value.

Further, “L_{A}^{3D}” in FIG. 11 indicates the degree of liberation of the component A as the three-dimensional state data, “L_{B}^{3D}” indicates the degree of liberation of the component B, and “Λ_{A}^{3D}” indicates the degree of locking focused on the component A. As illustrated in FIG. 11, those pieces of true value estimation data are obtained by correcting “L_{A}^{2D}” (degree of liberation of the component A in the two-dimensional state data), “L_{B}^{2D}” (degree of liberation of the component A in the two-dimensional state data), and “Λ_{A}^{2D}” (degree of locking of the component A in the two-dimensional state data), which are the two-dimensional state data, with “σ_{A}” being the stereological bias corrected value for the component A, “σ_{B}” being the stereological bias corrected value for the component B, and “Λ_{A}^{Dif}” corresponding to the stereological bias corrected value of the component A for the degree of locking.

Now, a supplementary description is given of the method of analyzing the degree of locking in relation to both of the three-dimensional state estimation device according to the first embodiment and the three-dimensional state estimation device according to the second embodiment.

The degree of locking indicates, as described above, any one of the area fraction, volume fraction, mass fraction, and count fraction in the group of locked particles in which any one of the area fraction, volume fraction, and mass fraction of the component of interest in one particle is a fixed fraction.

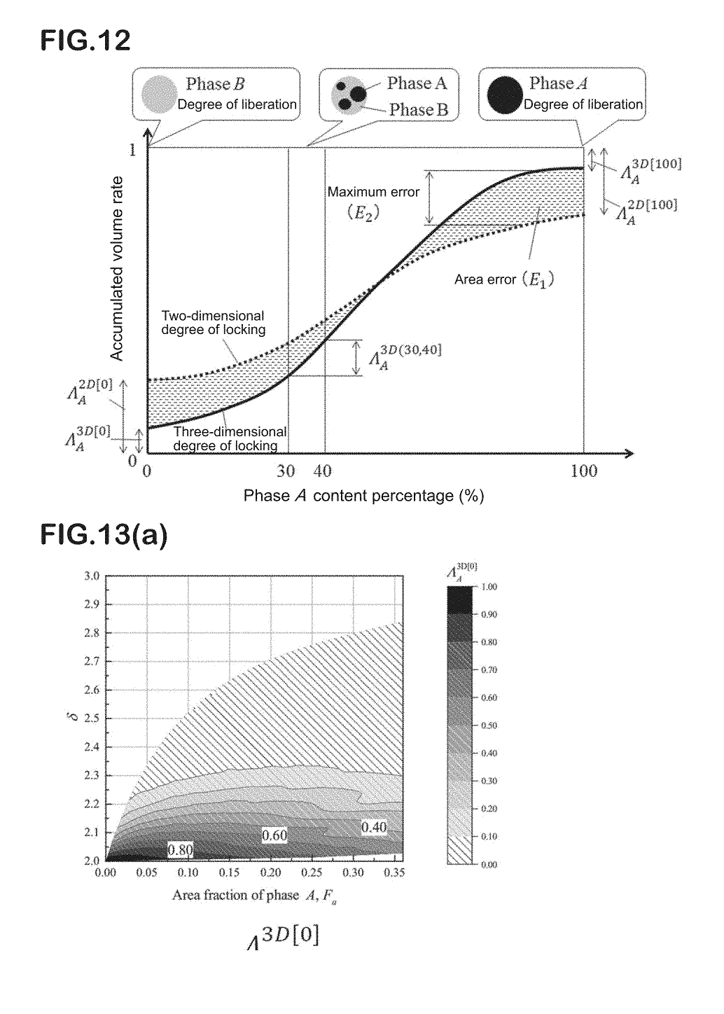

Now, as one example, the multicomponent material formed of two-component particles, namely, the component A and the component B are classified into 12 classes (0%, larger than 0% and smaller than 10%, larger than 10% and smaller than 20%, larger than 20% and smaller than 30%, larger than 30% and smaller than 40%, larger than 40% and smaller than 50%, larger than 50% and smaller than 60%, larger than 60% and smaller than 70%, larger than 70% and smaller than 80%, larger than 80% and smaller than 90%, larger than 90% and smaller than 100%, and 100%). A conceptual diagram of the degree of locking at this time is shown in FIG. 12.

The method of calculating the degree of locking (Λ_{A}^{3D}) shown in FIG. 12 comprises, for example, a method of geometrically calculating volume information and area information of two-component aggregated particles.

The volume information can be obtained by calculating, based on the coordinates and radii of the particles and the component A, a total volume (Vₐₗₗ) of the particles, a total volume (V_{A}^{lib}) of a region of the liberated component A, a total volume (V_{B}^{lib}) of a region of the liberated component B, and a total volume (V(x)) of particles whose volume fraction of the component A is x %.

Further, the area information can be obtained by calculating, for a freely-selected cross-section and based on the coordinates, radii, and heights of the cross-sections of the particles and the component A, a total area (Sₐₗₗ) of the cross-sections of the particles, a total area (S_{A}^{lib}) of the region of the apparently liberated component A, a total area (S_{B}^{lib}) of the domain of the apparently liberated component B, and a total area (S(x)) of particles whose volume fraction of the component A is x %.

More specifically, those pieces of data can be calculated based on the following Equations (39) to (43).

$$\begin{matrix}[ Numerical Equation 15 ] & \\ {A}_{A}^{3 D [ 0 ]} = \frac{V ( 0 )}{{V}_{all}} = \frac{{V}_{B}^{lib}}{{V}_{all}} = {L}_{B}^{3 D} ( 1 - {F}_{v} ) , & ( 39 ) \\ {A}_{A}^{3 D ( i , i + 10 )} = \frac{{\sum }_{i < x \leq i + 10} V ( x )}{{V}_{all}} , \\ i = 0 , 10 , 20 , 30 , 40 , 50 , 60 , 70 , 80 & ( 40 ) \\ {A}_{A}^{3 D ( 90 , 100 )} = \frac{{\sum }_{90 < x < 100} V ( x )}{{V}_{all}} & ( 41 ) \\ {A}_{A}^{3 D [ 100 ]} = \frac{V ( 100 )}{{V}_{all}} = \frac{{V}_{A}^{lib}}{{V}_{all}} = {L}_{A}^{3 D} {F}_{v} & ( 42 )\end{matrix}$$

Regarding the said Equation (41), for example, when i=10 is satisfied, the degree of locking (Λ_{A}_{^{3D}}) is calculated as follows.

$$\begin{matrix}[ Numerical Equation 16 ] & \\ {A}_{A}^{3 D ( 10 , 20 ]} = \frac{{\sum }_{10 < x \leq 20} V ( x )}{{V}_{all}} & ( 43 )\end{matrix}$$

Further, similarly to the degree of locking (Λ_{A}^{3D}), the two-dimensional degree of locking (Λ_{A}_{^{2D}}) is calculated as follows.

$$\begin{matrix}[ Numerical Equation 17 ] & \\ {A}_{A}^{2 D [ 0 ]} = \frac{1}{n} {\sum }_{1}^{n} \frac{S ( 0 )}{{S}_{all}} = \frac{1}{n} {\sum }_{1}^{n} \frac{{S}_{B}^{lib}}{{S}_{all}} = {L}_{B}^{2 D} ( 1 - {F}_{a} ) & ( 44 ) \\ {A}_{A}^{2 D ( i , i + 10 )} = \frac{1}{n} {\sum }_{1}^{n} \frac{{\sum }_{i < x \leq i + 10} S ( x )}{{S}_{all}} , \\ i = 0 , 10 , 20 , 30 , 40 , 50 , 60 , 70 , 80 & ( 45 ) \\ {A}_{A}^{2 D ( 90 , 100 )} = \frac{1}{n} {\sum }_{1}^{n} \frac{{\sum }_{90 < x < 100} S ( x )}{{S}_{all}} & ( 46 ) \\ {A}_{A}^{2 D [ 100 ]} = \frac{1}{n} {\sum }_{1}^{n} \frac{S ( 100 )}{{S}_{all}} = \frac{1}{n} {\sum }_{1}^{n} \frac{{S}_{A}^{lib}}{{S}_{all}} = {L}_{A}^{2 D} {F}_{a} & ( 47 )\end{matrix}$$

The mass fraction of the locked particle in the group of particles is calculated by replacing, in the said Equations (39) to (42), Vₐₗₗ with the total masses of those particles, V_{A}^{lib} with the total masses of particles each having the liberated component A, V_{B}^{lib} with the total masses of particles each having the liberated component B, V(x) with the total masses of particles whose volume fraction of the component A is x %.

Further, the count fraction of the locked particle in the group of particles is calculated by replacing, in the said Equations (39) to (42), Vₐₗₗ with the total number of particles, V_{A}^{lib} with the total number of particles each having the liberated component A, V_{B}^{lib} with the total number of particles each having the liberated component B, and V(x) with the total number of particles for which the volume fraction of the component A is x %.

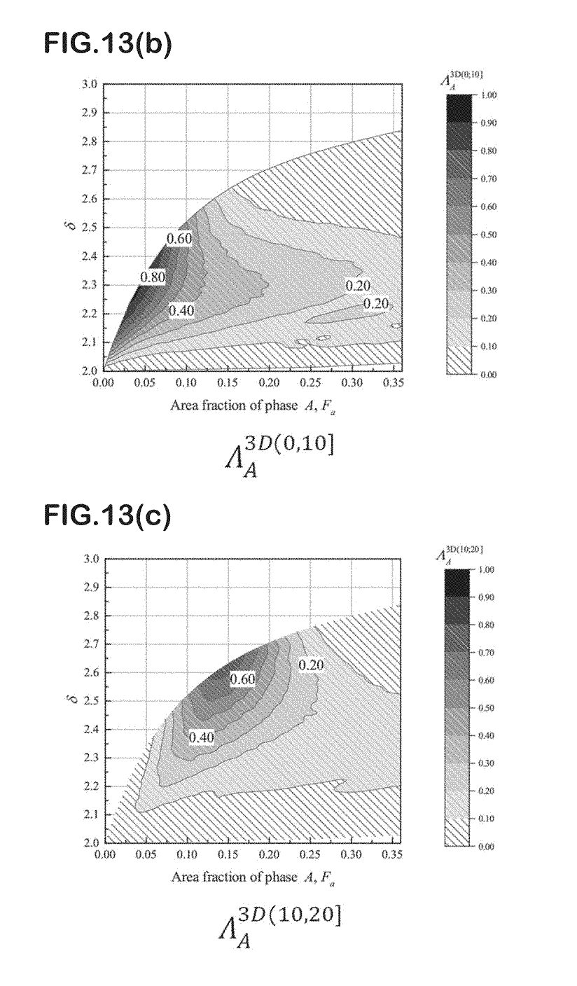

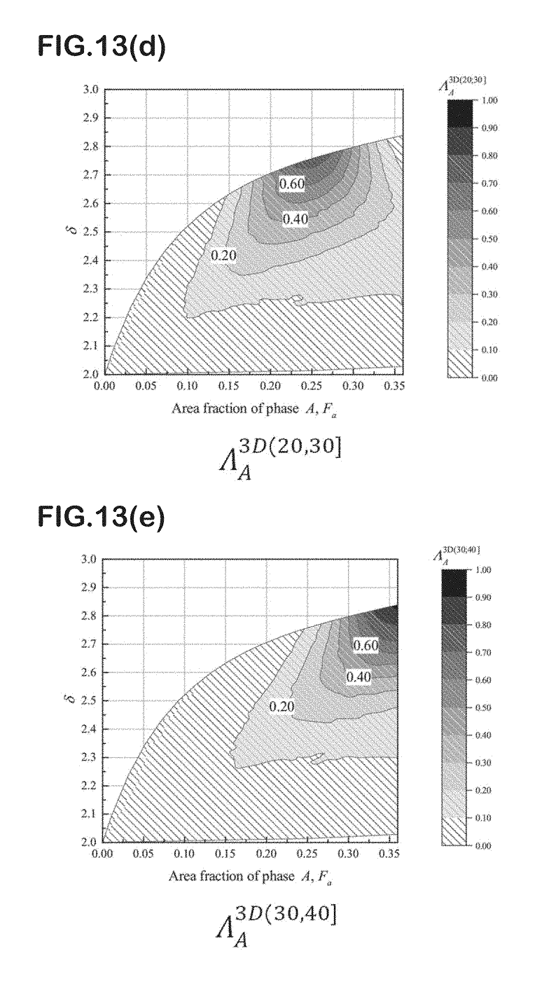

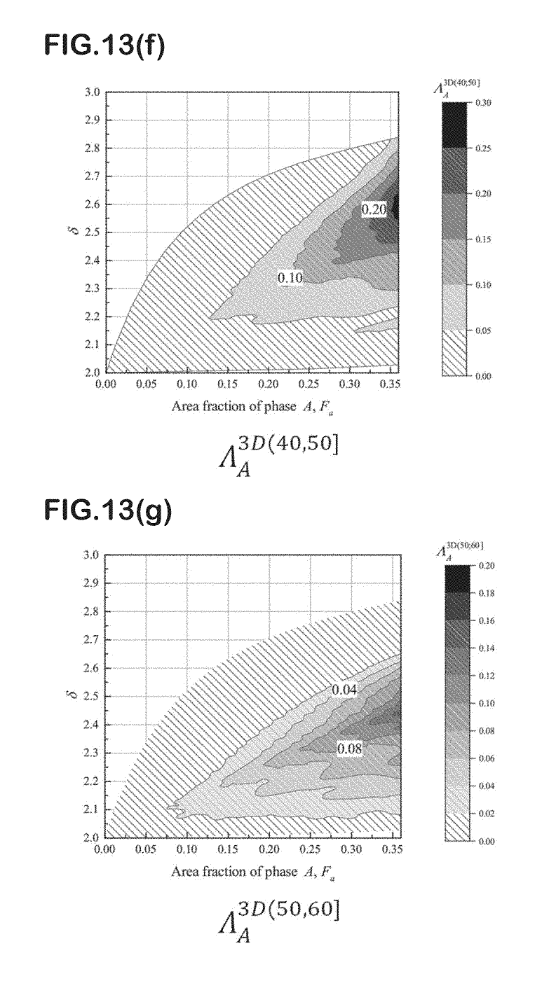

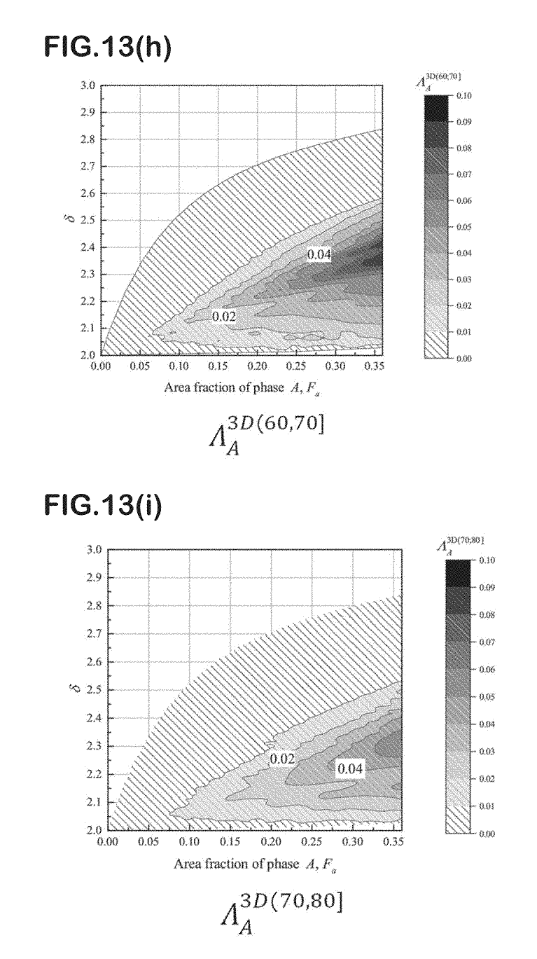

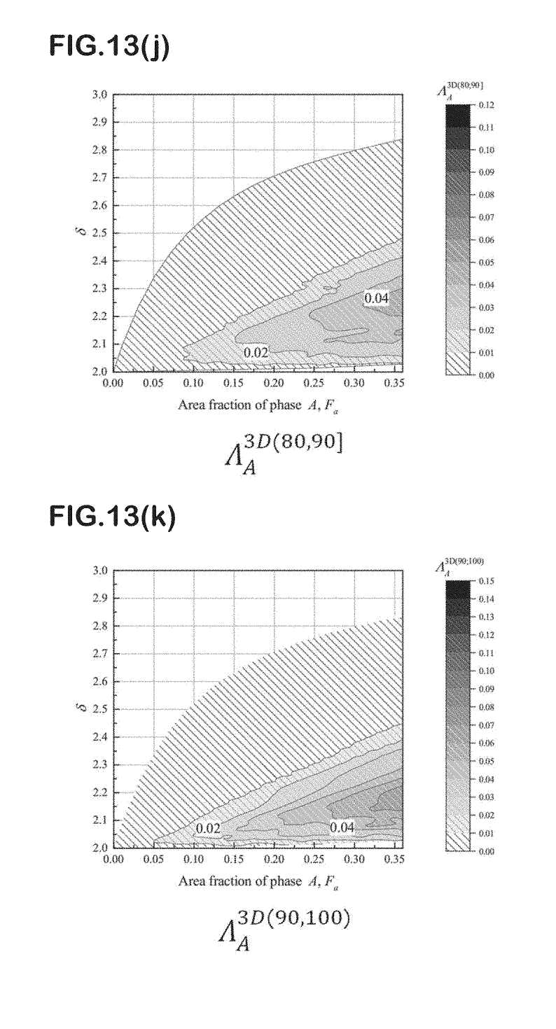

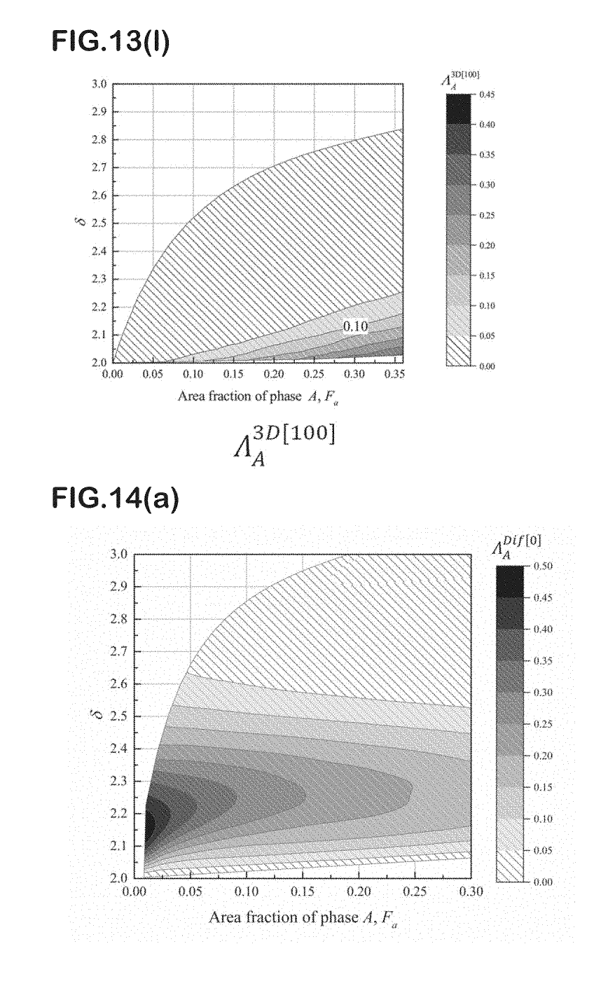

Examples of the contour line diagram of the statistical data, which has been obtained by taking statistics of the correlation among the fractal dimensional value δ, the area fraction Fa, and the degree of locking Λ_{A}^{3D}) through calculation of a sample image, are shown in FIG. 13(a) to FIG. 13(i). FIG. 13(a) to FIG. 13(i) are illustrations of the statistical data set by the statistical data setting means 1 in the three-dimensional state estimation device according to the first embodiment with the degree of locking (Λ_{A}^{3D}) of the component A being the three-dimensional state data. FIG. 13(a) is an illustration of a contour line diagram in a case where the content percentage of the component A is0%, FIG. 13(b) is an illustration of a contour line diagram in a case where the content percentage of the component A is larger than0% and equal to or smaller than 10%, FIG. 13(c) is an illustration of a contour line diagram in a case where the content percentage of the component A is larger than 10% and equal to or smaller than 20%, FIG. 13(d) is an illustration of a contour line diagram in a case where the content percentage of the component A is larger than 20% and equal to or smaller than 30%, FIG. 13(e) is an illustration of a contour line diagram in a case where the content percentage of the component A is larger than 30% and equal to or smaller than 40%, FIG. 13(f) is an illustration of a contour line diagram in a case where the content percentage of the component A is larger than 40% and equal to or smaller than 50%, FIG. 13(g) is an illustration of a contour line diagram in a case where the content percentage of the component A is larger than 50% and equal to or smaller than 60%, FIG. 13(h) is an illustration of a contour line diagram in a case where the content percentage of the component A is larger than 60% and equal to or smaller than 70%, FIG. 13(i) is an illustration of a contour line diagram in a case where the content percentage of the component A is larger than 70% and equal to or smaller than 80%, FIG. 13(j) is an illustration of a contour line diagram in a case where the content percentage of the component A is larger than 80% and equal to or smaller than 90%, FIG. 13(k) is an illustration of a contour line diagram in a case where the content percentage of the component A is larger than 90% and smaller than 100%, and FIG. 13(l) is an illustration of a contour line diagram in a case where the content percentage of the component A is 100%. Regarding Λ_{A}^{3D} illustrated in each of FIG. 13(a) to FIG. 13(l), a supersubscript, for example, [0] denotes a meaning similar to that of, for example, [0] in the said Equations (39) to (47).

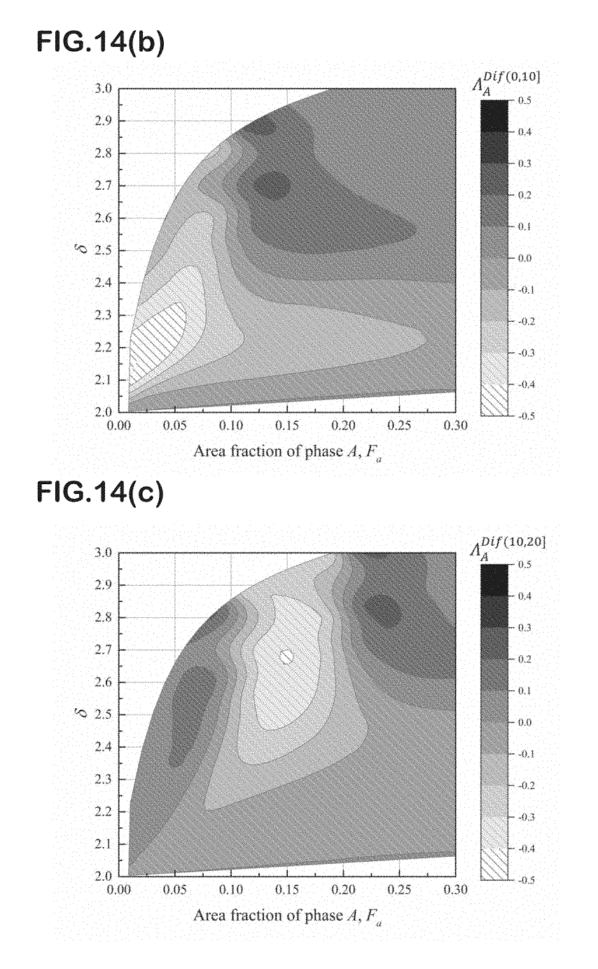

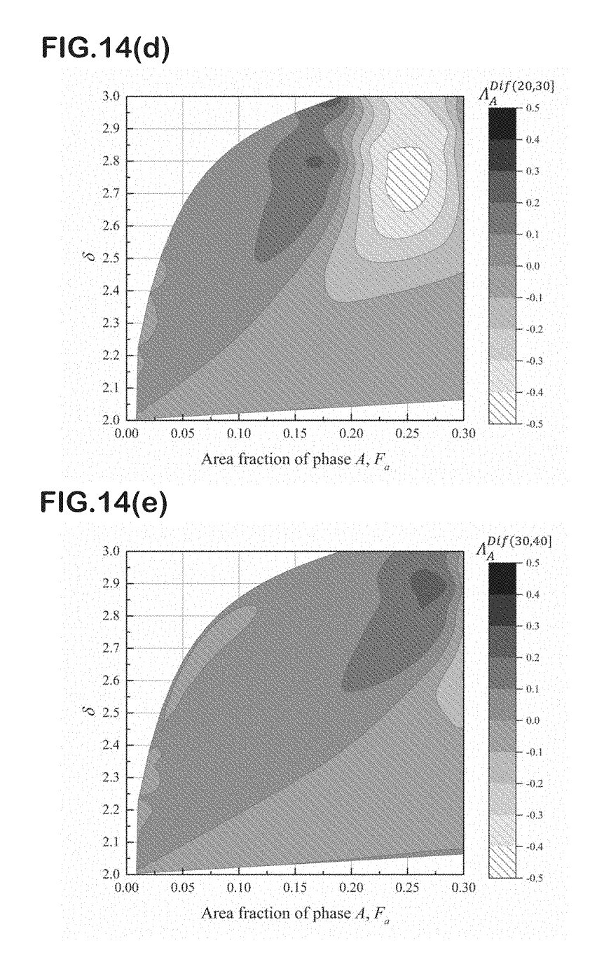

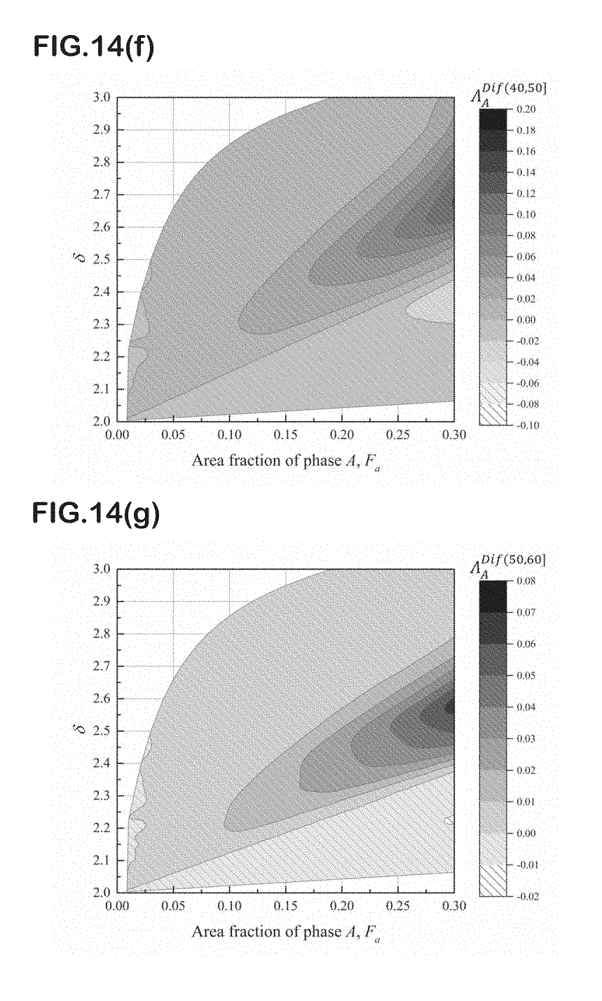

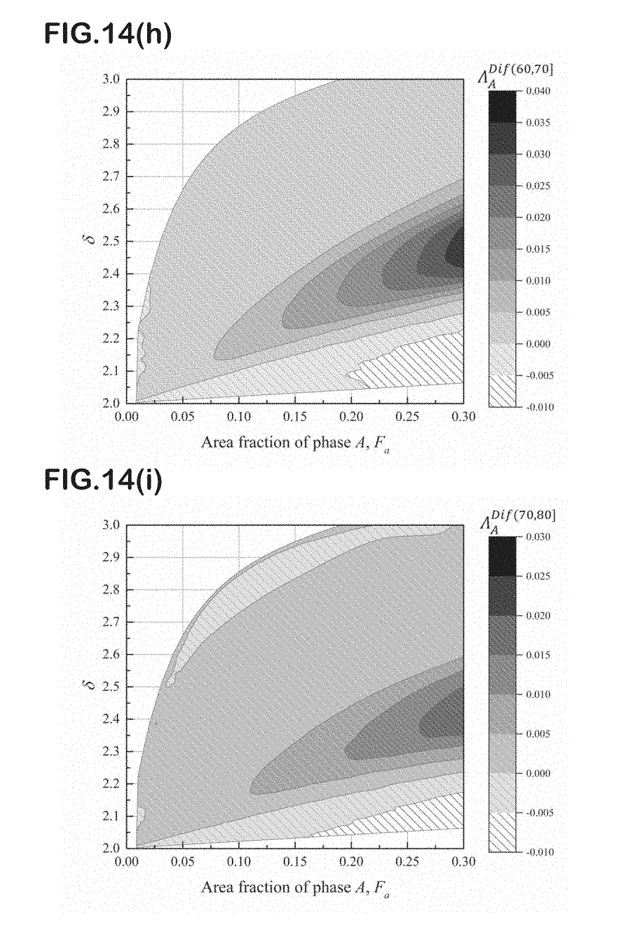

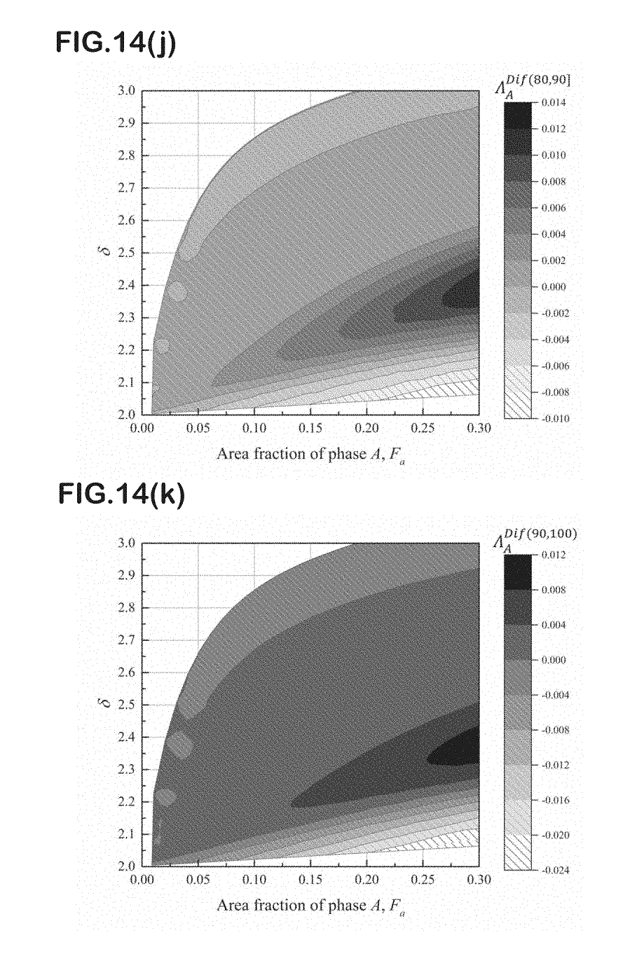

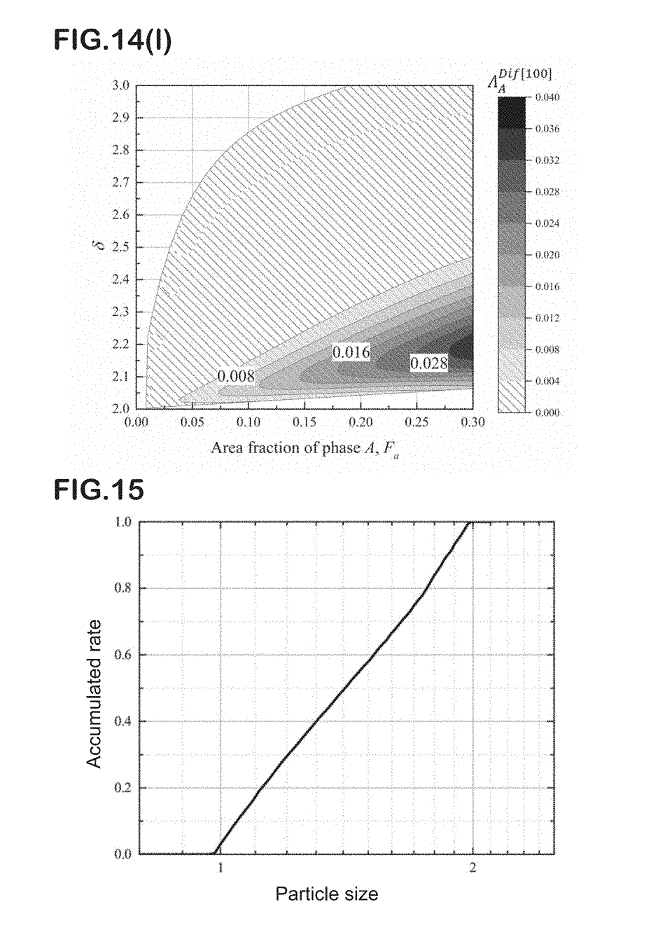

Examples of the contour line diagram of the statistical data, which has been obtained by taking statistics of the correlation among the fractal dimensional value δ, the area fraction Fa, and the stereological bias corrected value (A_{A}^{Dif}) of the component A for the degree of locking through calculation of a sample image, are shown in FIG. 14(a) to FIG. 14(l). FIG. 14(a) to FIG. 14(l) are illustrations of the statistical data set by the statistical data setting means 10 in the three-dimensional state estimation device according to the second embodiment with the stereological bias corrected value (Λ_{A}^{Dif}) of the component A for the degree of locking being the correction state data. FIG. 14(a) is an illustration of a contour line diagram in a case where the content percentage of the component A is 0%, FIG. 14(b) is an illustration of a contour line diagram in a case where the content percentage of the component A is larger than 0% and equal to or smaller than 10%, FIG. 14(c) is an illustration of a contour line diagram in a case where the content percentage of the component A is larger than 10% and equal to or smaller than 20%, FIG. 14(d) is an illustration of a contour line diagram in a case where the content percentage of the component A is larger than 20% and equal to or smaller than 30%, FIG. 14(e) is an illustration of a contour line diagram in a case where the content percentage of the component A is larger than 30% and equal to or smaller than 40%, FIG. 14(f) is an illustration of a contour line diagram in a case where the content percentage of the component A is larger than 40% and equal to or smaller than 50%, FIG. 14(g) is an illustration of a contour line diagram in a case where the content percentage of the component A is larger than 50% and equal to or smaller than 60%, FIG. 14(h) is an illustration of a contour line diagram in a case where the content percentage of the component A is larger than 60% and equal to or smaller than 70%, FIG. 14(i) is an illustration of a contour line diagram in a case where the content percentage of the component A is larger than 70% and equal to or smaller than 80%, FIG. 14(j) is an illustration of a contour line diagram in a case where the content percentage of the component A is larger than 80% and equal to or smaller than 90%, FIG. 14(k) is an illustration of a contour line diagram in a case where the content percentage of the component A is larger than 90% and smaller than 100%, and FIG. 14(l) is an illustration of a contour line diagram in a case where the content percentage of the component A is 100%. Regarding Λ_{A}^{Dif} illustrated in each of FIG. 14(a) to FIG. 14(l), a supersubscript, for example, [0] denotes a meaning similar to that of, for example, [0] in the said Equations (39) to (47).

(Three-Dimensional State Estimation Program)

A three-dimensional state estimation program according to the present invention is a program for causing a computer to function as statistical data setting means for setting statistical data obtained by taking statistics of a correlation among: a complexity indicator for quantitatively indicating, as an image complexity, various display states of a component of interest in two-dimensional image data in which a cross-section or surface of a multicomponent material comprising a group of particles formed of a liberated particle and a locked particle is displayed and the component of interest and a component of non-interest in the group of particles in the cross-section or surface are displayed differently from each other; an area fraction of the component of interest in the two-dimensional image data; and three-dimensional estimation data, which is any one of three-dimensional state data on a content percentage of the component of interest in the multicomponent material at a time when the complexity indicator and the area fraction are determined and correction data for correcting two-dimensional state data on the content percentage of the component of interest in the cross-section or surface of the multicomponent material to the three-dimensional state data. The type of computer is not particularly limited, and a publicly known arithmetic processing device can be used.

The matters already described in the three-dimensional state estimation device of the present invention can be similarly applied to the complexity indicator, the area fraction, the three-dimensional estimation data, the statistical data, the statistical data setting means, and other matters, and thus a redundant description thereof is omitted here.

(Three-Dimensional State Estimation Method)

A three-dimensional state estimation method according to the present invention is a method comprising a statistical data setting step of setting statistical data obtained by taking statistics of a correlation among: a complexity indicator for quantitatively indicating, as an image complexity, various display states of a component of interest in two-dimensional image data in which a cross-section or surface of a multicomponent material comprising a group of particles formed of a liberated particle and a locked particle is displayed and the component of interest and a component of non-interest in the group of particles in the cross-section or surface are displayed differently from each other; an area fraction of the component of interest in the two-dimensional image data; and three-dimensional estimation data, which is any one of three-dimensional state data on a content percentage of the component of interest in the multicomponent material at a time when the complexity indicator and the area fraction are determined and correction data for correcting two-dimensional state data on the content percentage of the component of interest in the cross-section or surface of the multicomponent material to the three-dimensional state data.

The matters already described in the three-dimensional state estimation device of the present invention can be similarly applied to the complexity indicator, the area fraction, the three-dimensional estimation data, the statistical data, the statistical data setting step, and other matters, and thus a redundant description thereof is omitted here.

### EXAMPLES

(Setting of Statistical Data)

A multicomponent material formed of spherical particles was assumed, and statistical data was set as follows.

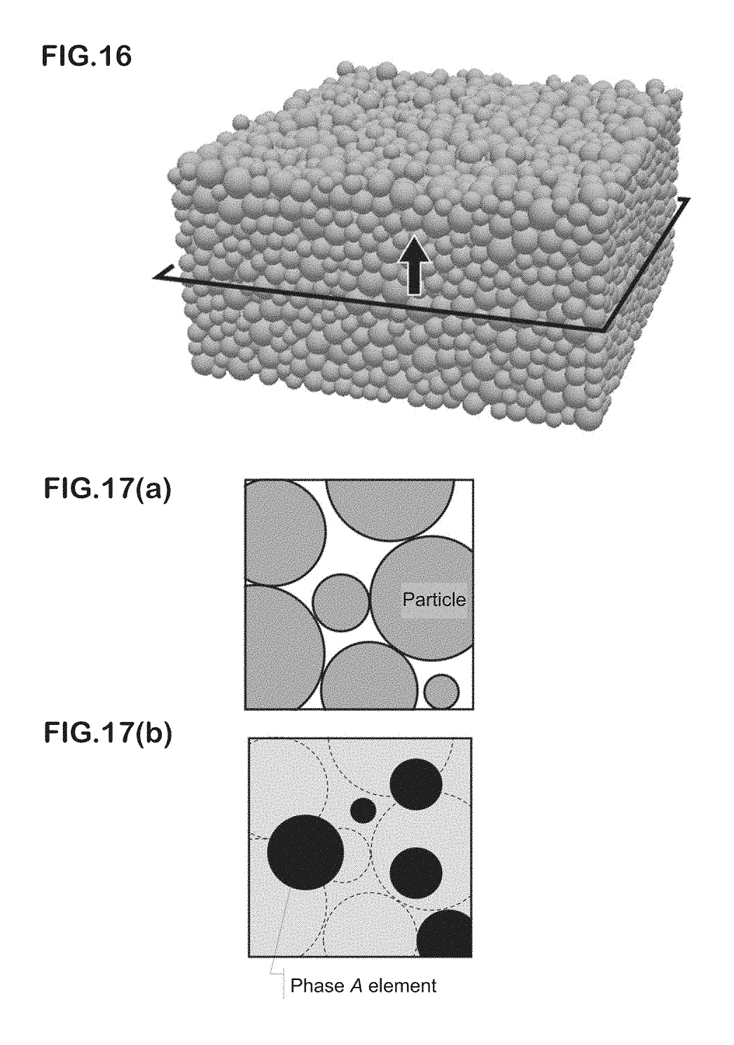

First, a distinct element method or discrete element method (DEM) described in the following Reference Document 2 given below is used to model a spherical element. The particle size (particle diameter) was set dimensionless, and 7,463 spherical particles that followed a particle size distribution shown in FIG. 15 were generated at random positions in a square region (width: 30, depth: 30, and height: 20). After that, the spherical particles were caused to freely drop in the square region, and aggregated spherical particles illustrated in FIG. 16 were created. FIG. 15 is a diagram for illustrating the particle size distribution of the spherical particles, and FIG. 16 is a diagram for illustrating the created aggregated spherical particles.
Reference Document 2: A. Cundall, P., L. Strack, O., D., A discrete numerical model for granular assemblies, Geotechnique. 29 (1979) 47-65.

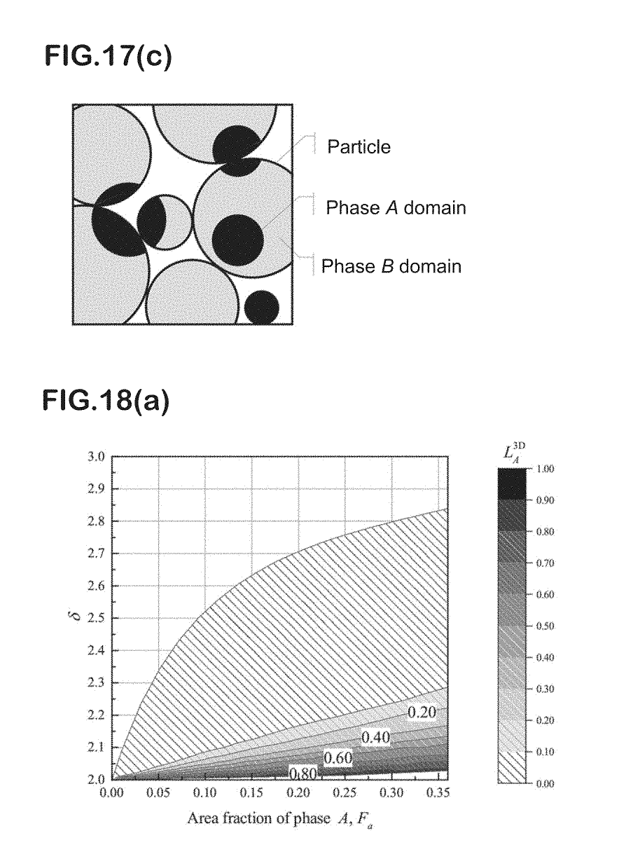

Next, domains of phase A and phase B components are set in the spherical particles. An outline of a method of creating two-component particles is illustrated in FIG. 17(a) to FIG. 17(c). FIG. 17(a) is a diagram for illustrating the spherical particles created by the distinct element method. A spherical element (referred to as “phase A element”) was generated at a random position in the same square region in which the spherical particles were generated (refer to FIG. 17(b)). FIG. 17(b) is a diagram for illustrating a state of generation of the phase A elements in the spherical elements. At this time, the phase A element was generated independently of the spherical particle, and thus it was assumed that the phase A element was able to exist at a position overlapping with the spherical particle. 3,726 patterns of the distribution of phase A elements were set, and details thereof are described later. The spherical particle and the phase A element were caused to overlap with each other to set a domain of the spherical particle overlapping with the phase A element as a phase A domain and other domains in the spherical particle as a phase B domain, to thereby create a spherical two-component particle (FIG. 17(c)). FIG. 17(c) is a diagram for illustrating the created spherical two-component particles.

The phase A element was set similarly to a spherical particle with the same particle size in 82 patterns of the particle size of from 0.40 to 2.00 in units of 0.02and in 46 patterns of the particle size of from 2.00 to 20.0 in units of 0.4. The volume fraction of the phase A element in the square region in this state was from about 0.30 to about 0.36.

Next, the phase A element was deleted randomly one by one to set various volume fractions, the volume fraction was repeatedly re-calculated, and a state of the volume fraction decreasing by 0.01 was recorded as an individual case. When the phase A element was large, simply deleting one phase A element resulted in decrease of the volume fraction by more than 0.01 in some cases, and in that case, decrease of the volume fraction by more than 0.01 was allowed. Such an operation was repeated to set 3,726 patterns of the phase A element with various particles sizes and volume fractions.

The volume information and area information on the aggregated particles (multicomponent material) relating to the two-component particle set as described above were geometrically calculated.

First, the volume information was obtained by calculating, based on the coordinates and radii of the spherical particles and the phase A element, a total volume (Vₐₗₗ) of the spherical particles, a total volume (V_{A}) of the phase A domain, a total volume (V_{B}) of the phase B domain, a total volume (V_{A}^{lib}) of the liberated phase A domain, and a total volume (V_{B}^{lib}) of the liberated phase B domain.

Further, the area information was obtained by calculating, for a freely-selected cross-section and based on the coordinates, radii, and heights of the cross-sections of the spherical particles and the phase A element, a total area (Sₐₗₗ) of the cross-sections of the spherical particles, a total area (S_{A}) of the phase A domain, a total area (S_{B}) of the phase B domain, a total area (S_{A}^{lib}) of the apparently liberated phase A domain, and a total area (S_{B}^{lib}) of the apparently liberated phase B domain. Information on the cross-sections of the spherical particles was calculated by setting the cross-sections as surfaces parallel to the bottom surface of the square region and using n pieces of cross-section information through equal division of heights 6 to 12 into n−1 blocks. In this case, the number (n) of cross-sections is set to 20.

Next, regarding the aggregated particles, the area fraction (Fa) of the phase A, the apparent degree of liberation (L_{A}^{2D}, L_{B}^{2D}) of the phase A and the phase B in a two-dimensional state, the volume fraction (F_{V}) of the phase A domain, and the degree of liberation (L_{A}^{3D}, L_{B}^{3D}) of the phase A and the phase B in the three-dimensional state were defined and calculated as follows.

$$\begin{matrix}[ Numerical Equation 18 ] & \\ {F}_{a} = \frac{1}{n} {\sum }_{1}^{n} \frac{{S}_{A}}{{S}_{all}} & ( 48 ) \\ {L}_{A}^{2 D} = \frac{1}{n} {\sum }_{1}^{n} \frac{{S}_{A}^{lib}}{{S}_{A }} , & ( 49 ) \\ {L}_{B}^{2 D} = \frac{1}{n} {\sum }_{1}^{n} \frac{{S}_{B}^{lib}}{{S}_{B }} , & ( 50 ) \\ {F}_{v} = \frac{{V}_{A}}{{V}_{all}} & ( 51 ) \\ {L}_{A}^{3 D} = \frac{{V}_{A}^{lib}}{{V}_{A}} & ( 52 ) \\ {L}_{B}^{3 D} = \frac{{V}_{B}^{lib}}{{V}_{B}} & ( 53 )\end{matrix}$$

In principle of the stereological bias, L_{A}^{2D}≥L_{A}^{3D} and L_{B}^{2D}≥L_{B}^{3D} are satisfied.

Further, the degree-of-liberation over-estimation rate (σ_{A}, σ_{B}) is defined as follows in order to quantitatively evaluate the influence of a stereological bias in the apparent degree of liberation in the two-dimensional state.

$$\begin{matrix}[ Numerical Equation 19 ] & \\ {σ}_{A} = \frac{{L}_{A}^{2 D} - {L}_{A}^{3 D}}{{L}_{A}^{2 D}} & ( 54 ) \\ {σ}_{B} = \frac{{L}_{B}^{2 D} - {L}_{B}^{3 D}}{{L}_{B}^{2 D}} & ( 55 )\end{matrix}$$

Further, the fractal dimension (δ) was calculated as the complexity indicator with a method similar to that described with reference to FIG. 5.

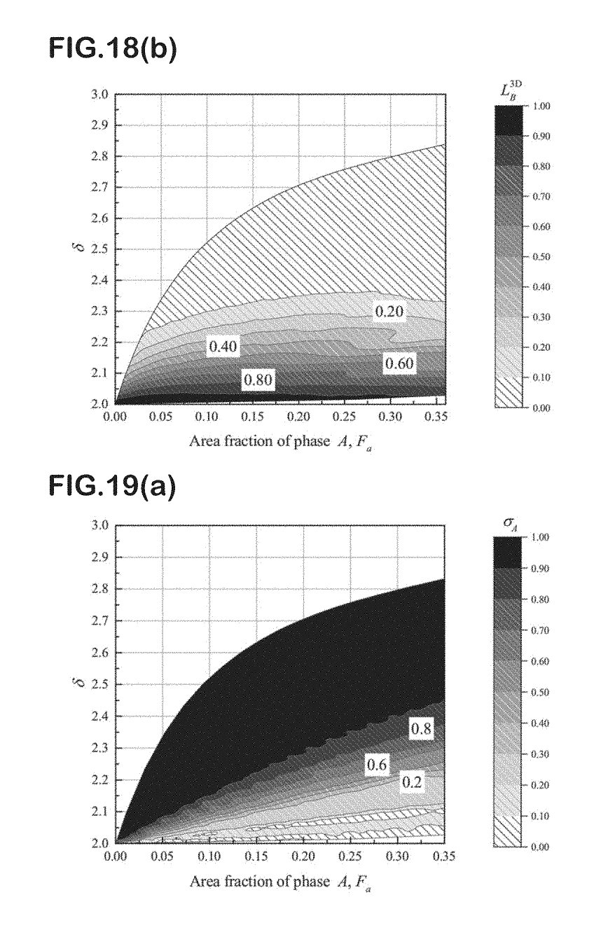

Contour line diagrams created by plotting the degree of liberation (L_{A}^{3D}, L_{B}^{3D}) in the analyzed three-dimensional state with respect to the fractal dimension (δ) and the area fraction (Fa) are shown in FIG. 18(a) and FIG. 18(b). FIG. 18(a) is a contour line diagram obtained by taking statistics of the correlation among the degree of liberation (L_{A}^{3D}), the fractal dimension (δ), and the area fraction (Fa). FIG. 18(b) is a contour line diagram obtained by taking statistics of the correlation among the degree of liberation (L_{B}^{3D}), the fractal dimension (δ), and the area fraction (Fa).

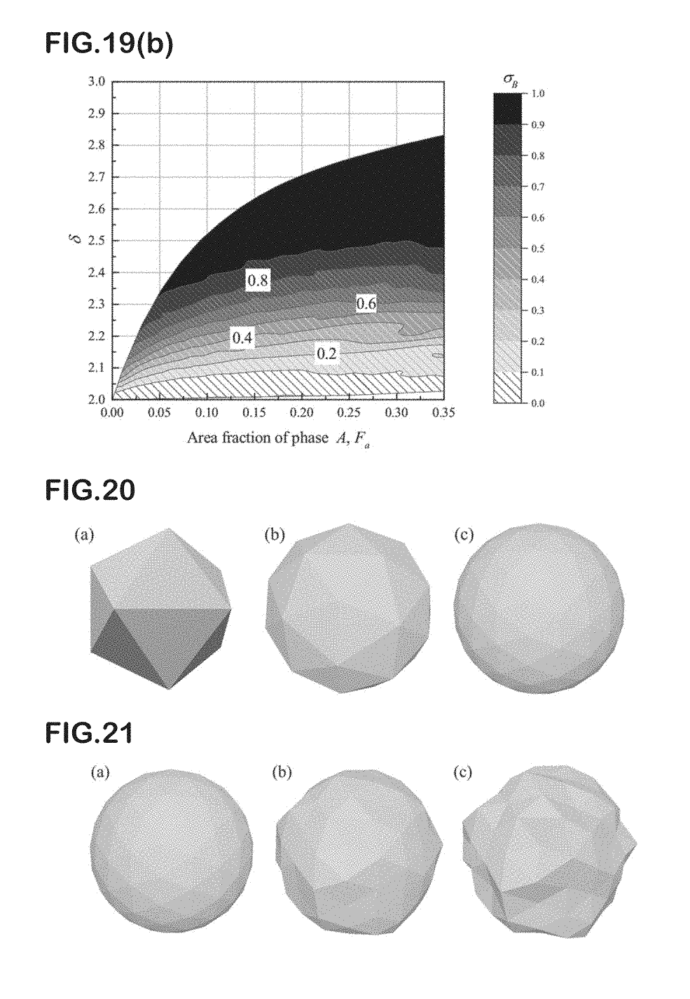

Further, contour line diagrams created by plotting the analyzed degree-of-liberation over-estimation rate (σ_{A}, σ_{B}) with respect to the fractal dimension (δ) and the area fraction (Fa) are shown in FIG. 19(a) and FIG. 19(b). FIG. 19(a) is a contour line diagram obtained by taking statistics of the correlation among the degree-of-liberation over-estimation rate (σ_{A}), the fractal dimensional value (δ), and the area fraction (Fa). FIG. 19(b) is a contour line diagram obtained by taking statistics of the correlation among the degree-of-liberation over-estimation rate (σ_{B}), the fractal dimensional value (δ), and the area fraction (Fa).

The contour line diagrams illustrated in FIG. 18(b) and FIG. 19(b) are created by setting the horizontal axis as the area fraction (Fa) of the phase A. However, the area fraction of the phase B is represented by (1−Fa), and thus the contour line diagram can also be created by the area fraction of the phase B depending on a component of interest.

The statistical data was set as described above.

(Multicomponent Material Serving as Estimation Target)

Next, a description is given of a multicomponent material serving as the estimation target.

The real multicomponent material, for example, a natural ore is originally used as the estimation target, but in this Example, the virtually set multicomponent material is used for the following reasons.

Specifically, when the real multicomponent material is used, an accuracy of a true value of, for example, the three-dimensional state data, is less likely to be ensured due to a measurement error. Thus, the multicomponent material is virtually set to ensure the accuracy of a true value of, for example, the three-dimensional state data on the multicomponent material, accurately perform verification of the estimation error of the true value estimation data as viewed from the true value, and clarify effectiveness of the estimation processing in the present invention.

Meanwhile, the multicomponent material is virtually set as follows in such a manner as to accurately simulate the real multicomponent material in order to prevent deviation from the real multicomponent material.

First, an aspect ratio (α) representing the fineness of a particle and a corrected sphericity (S_{c}) representing smoothness of the surface are used to define the aspect ratio (α) and the corrected sphericity (Sₑ) as follows.

$$\begin{matrix}[ Numerical Equation 20 ] & \\ α = \frac{α}{c} & ( 56 ) \\ {S}_{c} = \frac{{A}_{c}}{A} & ( 57 )\end{matrix}$$

In the said Equations (56) and (57), “a” represents a major-axis length of the particle, “c” represents a minor-axis length of the particle, “A” represents a surface area of the particle, and “Ac” represents a surface area of an ellipsoid with the same volume and aspect ratio as those of the particle.

A technique of segmentalizing (subdividing), for example, a ball with polyhedrons, which is called a geodesic grid method, is applied to a method of modeling with distorted shapes the various particles to which the aspect ratio (α) and the corrected sphericity (S_{c}) are set.

First, an icosahedron inscribed in the spherical particle serving as a model is created, and set as a first-generation particle (refer to FIG. 20(a)). Next, a midpoint of each side of the icosahedron is projected onto the spherical particle to create a second-generation particle with fine meshes (refer to FIG. 20(b)). This operation is repeated to enable creation of a polyhedron with fine meshes. A third-generation particle illustrated in FIG. 20(c) is used as the model particle. FIG. 20 are explanatory diagrams for illustrating the particle created by the geodesic grid method.

Further, an elliptical particle before application of the geodesic grid method was used to perform processing similar to that for the spherical particle described above.

In this manner, the model particle was set to the spherical particle and the elliptical particle with different aspect ratios.

Next, a nodal point of the third-generation particle was dispersed in a direction of the gravity of particles toward the nodal point by the following value.

$$\begin{matrix}[ Numerical Equation 21 ] & \\ Dispersion = \frac{α ɛ γ}{2} & ( 58 )\end{matrix}$$

In the said Equation (58), ε indicates a uniform random number in a range of from −1 to 1, and γ indicates a parameter with the magnitude of dispersion.

Regarding the third-generation particle, an example of the particle having a sphere as its basic shape in a case where γ indicates 0.0 (no dispersion), 0.5, and 1.0 is illustrated in FIG. 21.

In this description, a particle whose corrected sphericity indicating smoothness of the particle surface takes various values is created by changing the value of γ.

In this manner, the shape of the model particle was set.

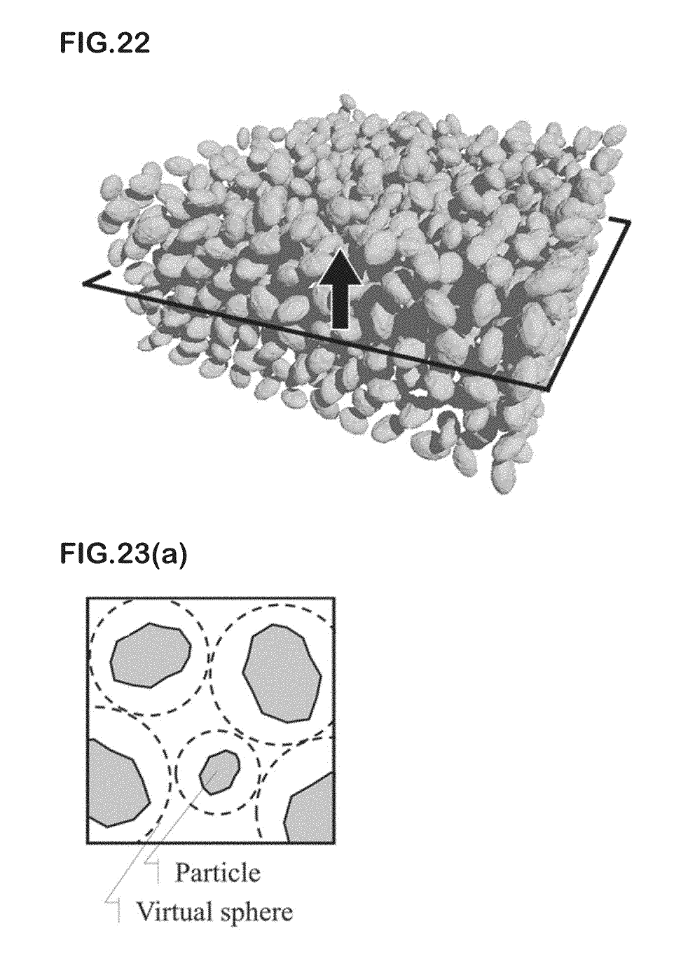

Next, the internal structure of the model particle is set, and at the same time, a multicomponent material formed of the model particles is set. Specifically, two components of the phase A and the phase B are set to the model particle as follows, and a multicomponent material formed of the model particles illustrated in FIG. 22 is set.

First, 7,463 virtual particles (virtual spheres) with a diameter (dᵥ) of from 1.0 to 2.0 were generated at random positions in a rectangular sample cell (W30, D30, H20), and then caused to freely drop to be packed, so that aggregated particles each being the virtual particle were set.

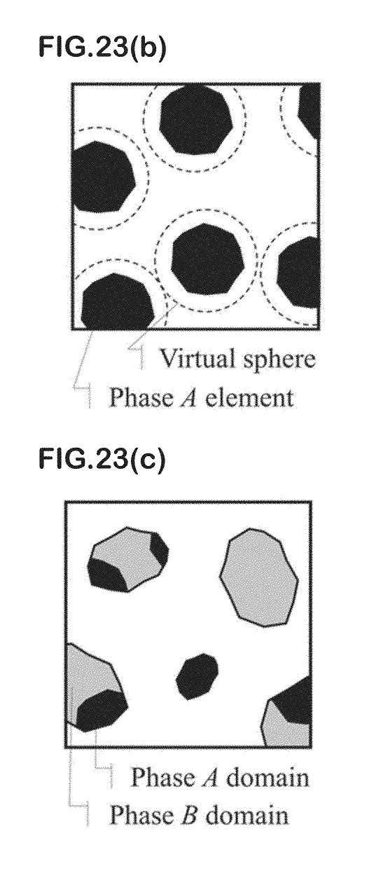

Next, the model particles (particles) modeled as the third-generation particles were generated around the virtual particles in random directions (refer to FIG. 23(a)). At this time, the size of each model particle was set in such a range that the model particle did not stick out from the virtual particle.

Next, the size of each first-generation particle was set with respect to the virtual particle in such a range that the first-generation particle did not stick out from the virtual particle, and the first-generation particles were generated in random directions. After that, a core component forming the phase A was set (refer to FIG. 23(b)).

Next, the model particle and the core component were caused to overlap with each other, and a part overlapped by the model particle and the core component was set as the phase A domain, and other domains were set as the phase B domain (refer to FIG. 23(c)).

The phase A and the phase B set for the model particle correspond to the phase A and the phase B of the spherical particle used for setting the statistical data.

Further, for the sake of convenience, in the following, the phase A before cutting out of the particle is referred to as the “element A”, and the phase A after cutting out of the particle is referred to as the “phase A” in a distinctive manner.

Further, FIG. 23(a) to FIG. 23(c) are explanatory diagrams for illustrating a method of setting two components of the phase A and the phase B for the model particle.

In this manner, the multicomponent material (refer to FIG. 22) formed of the model particles serving as the estimation target in Example was set.

Cross-section information on surfaces of the multicomponent material set in this manner, which are parallel to the bottom surface of the sample cell, is calculated by a technique of using a Monte Carlo method and solid-angle calculation in combination. At this time, in comparison between two-dimensional cross-section information and three-dimensional information, the cross-section has an individual intrinsic error, and thus it is required to pay attention to the fact that the stereological bias and the intrinsic error are exhibited in a combined manner. In order to reduce the intrinsic error, it is desired that as many cross-sections as possible be calculated, but this results in a large calculation load. In view of this, information on 20 cross-sections is calculated for one case as the number of cross-sections to which the central limit theorem can be applied in consideration of a balance between the calculation load and the validity of statistical processing.

The area and volume of the phase A are estimated by the Monte Carlo method and the solid-angle calculation for the model particle in the two-dimensional cross-section and the model particle in the three-dimensional state.

The Monte Carlo method is a technique of plotting a large number of points at random positions in a region of a fixed volume (or area) containing particles (or particle cross-sections), and estimating the volumes (or areas) of those particles (or particle cross-sections) based on a ratio of the number of points contained in the particles (or particle cross-sections) to the number of entire points.

In this description, as the reference number of plots, 20,000 plots were adopted for calculation of the area of the model particle in the two-dimensional cross-section, and 80,000 plots were adopted for calculation of the volume of the model particle in the three-dimensional state.

In three-dimensional calculation, 80,000 plots were formed in a cube in which the virtual particles were inscribed to estimate the volume.

Further, in two-dimensional calculation, the size of the cross-section of the model particle varies depending on the position of the model particle to be cut. Thus, in order to implement estimation by the number of plots that depends on the cross-sectional area, the radius of a cross-section of the virtual particle in one section was set to r1, and the radius of the virtual particle itself was set to r2 so as to form 20,000*(r1/r2)² plots in a cube in which a circle of the radius r1 was inscribed to estimate the area.

At the time of determination of whether each plot obtained by the Monte Carlo method is inside or outside of, for example, the model particle, when, for example, the model particle is an ellipsoid (comprising sphere), whether each plot is inside or outside of the model particle can be determined by using an ellipsoid formula. However, the model particle has a distorted shape, and thus a technique of determining whether each plot is inside or outside of the model particle by dividing the surface of, for example, the model particle with meshes and calculating the solid angle was used. The solid angle (Ω) of a closed surface is calculated by the following Equation.

$$\begin{matrix}[ Numerical Equation 22 ] & \\ Ω = {\int }_{S} \frac{t \cdot n}{{t}^{2}} dS = \{ \begin{matrix}0 & ( when the plot is outside S ) \\ 4 π & ( when the plot is inside S )\end{matrix} & ( 59 )\end{matrix}$$

In the said Equation (59), S represents the closed surface of the model particle, t represents the size of a position vector in a minute region on the surface of the model particle. In the said Equation (59), bold t indicates a unit position vector in the minute region, and n indicates a normal vector in the minute region.

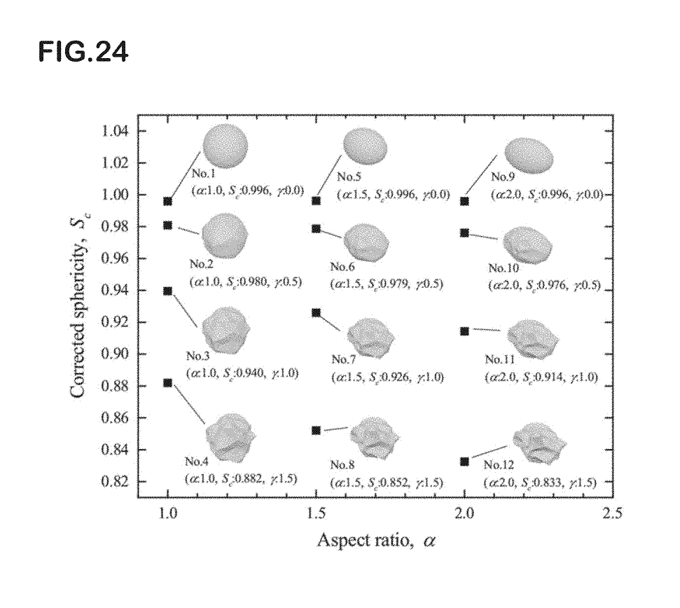

As described above, the model particle used for analysis was basically the third-generation particle, and as illustrated in FIG. 24, the aspect ratio (α) was set to three patterns of 1.0, 1.5, and 2.0, γ was set to four patterns of 0.0, 0.5, 1.0, and 1.5, and the corrected sphericity (S_{c}) was set to 12 patterns in a distributed manner within a range of from 0.83 to 1.00. FIG. 24 is a diagram for illustrating 12 patterns of the model particle.

The ratio between the major-axis length (a) and the minor-axis length (c) was obtained from the aspect ratio (α), and the middle-axis length (b) was obtained from a geometric mean (√ac) of the major-axis length (a) and the minor-axis length (c).

Further, in the case of the aspect ratio being 2.0, the size of the model particle was calculated so that the major-axis length (a) was equal to the diameter (dᵥ) of the spherical virtual particle, and the middle-axis length (b) and the minor-axis length (c) were also calculated from the aspect ratio (=2.0).

The major-axis length (a), the middle-axis length (b), and the minor-axis length (c) in a case where the aspect ratio was 1.0 or 1.5 were set so as to achieve the same particle volume as a case of the same condition in which the aspect ratio is 2.0 and so that the aspect ratio satisfies 1.0 and 1.5.

The core component (the element A) is basically the first-generation particle, and the aspect ratio (α) and γ are set to 1.0 and 0.0, respectively. Regarding the core component (the element A), the diameter (dv) of the virtual particle was set to 1.20, the percentage of the total volume (refer to FIG. 23(b)) of the core component (the element A) to the volume of a square region was set to 0.152, and the number of elements of the core component (the element A) was set to 1,290.

On the basis of the setting described above, the true values of the degree of liberation in the two-dimensional state, the degree of liberation in the three-dimensional state, and the degree-of-liberation over-estimation rate of the multicomponent material with distorted particle shapes and serving as the estimation target were calculated.

Further, regarding the multicomponent material with distorted particle shapes, the fractal dimension value (δ) and the area fraction (Fa), which are pieces of data to be input to the estimation processing, were calculated by a processing method similar to that of the processing of calculating the two-dimensional image data on the spherical particle to which the statistical data was set.

All the calculation processing described above was performed for the phase B in accordance with the calculation processing for the phase A.

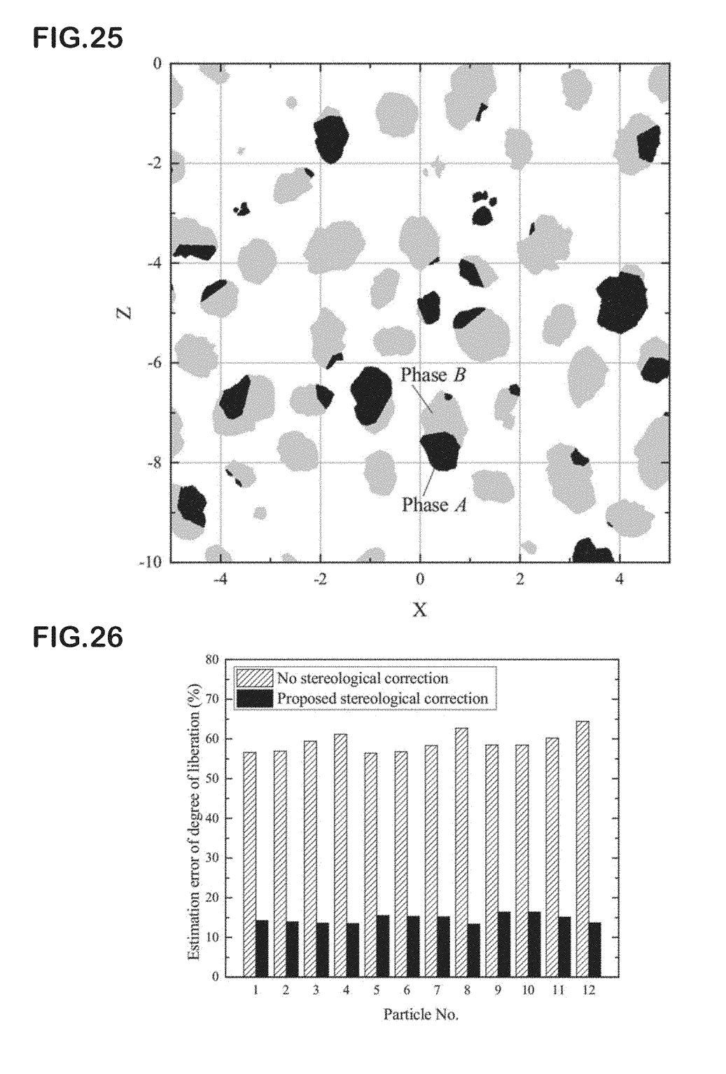

Further, as an example of the analysis result, FIG. 25 is an illustration of a cross-section of the multicomponent material created by the model particle (α=2.0, Sc=0.914) of No. 11. As illustrated in FIG. 23(a) to FIG. 23(c), it is to be understood that the locked particles and particles apparently liberated due to the phase A and the phase B are randomly generated.

(Direct Estimation of True Value Estimation Data based on Three-dimensional State Data)

Now, a description is given with the phase B serving as the component of interest. Further, a description is given of all the 12 patterns of the multicomponent material formed of 12 types particles illustrated in FIG. 24.

The contour line diagram illustrated in FIG. 18(b) set based on the multicomponent material formed of the spherical particles was used as the statistical data for collation with input data on the fractal dimension value (δ) and the area fraction (Fa) of the multicomponent material with distorted particle shapes serving as the estimation target to read, from the contour line diagram, the degree of liberation in the three-dimensional state corresponding to the fractal dimension value (δ) and the area fraction (Fa), and the degree of liberation is directly set as the true value estimation data (L_{R}^{3D′}) for the degree of liberation of the estimation target in the three-dimensional state.

As a comparative example of the present invention, the degree of liberation (L_{B}^{2D}) in the two-dimensional state corresponding to the fractal dimension value (δ) and the area fraction (Fa) of the multicomponent material with distorted particle shapes serving as the estimation target is set as comparison data.

The fractal dimension value (δ) and the area fraction (Fa) of the multicomponent material with distorted particle shapes, the degree of liberation (L_{B}^{2D}) in the two-dimensional state, the true value estimation data (L_{B}^{3D′}) for the degree of liberation of the estimation target in the three-dimensional state, and the true value (L_{B}^{3D}) for the degree of liberation of the estimation target in the three-dimensional state, which was calculated from the virtual setting of the multicomponent material with distorted particle shapes, are illustrated in Table 1 shown below.

TABLE 1

| Type of | | | | | |
|---|---|---|---|---|---|
| particle | Fₐ | δ | L_{B}^{3D′} | L_{B}^{3D} | L_{B}^{2D} |
| 1 | 0.151 | 2.213 | 0.350 | 0.408 | 0.639 |
| 2 | 0.151 | 2.213 | 0.350 | 0.407 | 0.638 |
| 3 | 0.151 | 2.215 | 0.345 | 0.399 | 0.637 |
| 4 | 0.151 | 2.217 | 0.340 | 0.393 | 0.633 |
| 5 | 0.151 | 2.215 | 0.345 | 0.408 | 0.639 |
| 6 | 0.151 | 2.215 | 0.345 | 0.407 | 0.638 |
| 7 | 0.151 | 2.217 | 0.340 | 0.401 | 0.635 |
| 8 | 0.152 | 2.220 | 0.335 | 0.387 | 0.629 |
| 9 | 0.152 | 2.219 | 0.335 | 0.401 | 0.635 |
| 10 | 0.152 | 2.219 | 0.335 | 0.401 | 0.635 |
| 11 | 0.152 | 2.219 | 0.335 | 0.395 | 0.632 |
| 12 | 0.152 | 2.222 | 0.330 | 0.382 | 0.629 |

Next, in order to check the validity of the estimation processing in the present invention, the true value estimation data (L_{B}^{3D′}) for the degree of liberation of the estimation target in the three-dimensional state was substituted into Lest of the following Equation (60) to calculate the estimation error of the degree of liberation (E) for the true value (L_{B}^{3D}), the degree of liberation (L_{B}^{2D}) in the two-dimensional state was substituted into L_{B}^{est} of the following Equation (60) to calculate the estimation error of the degree of liberation (E) for the true value (L_{B}^{3D}), and the estimation errors of the degree of liberation (E) were compared with each other.

$$\begin{matrix}[ Numerical Equation 23 ] & \\ E = 100 \frac{\vert {L}_{B}^{3 D} - {L}_{B}^{est} \vert}{{L}_{B}^{3 D}} & ( 60 )\end{matrix}$$

FIG. 26 is a graph for showing a comparison between the estimation error of the degree of liberation (E) for the true value (L_{B}^{3D}) that is obtained by substituting the true value estimation data (L_{B}^{3D′}) for the degree of liberation of the estimation target in the three-dimensional state into L_{B}^{est} and the estimation error of the degree of liberation (E) for the true value (L_{B}^{3D}) that is obtained by substituting the degree of liberation (L_{B}^{2D}) of the estimation target in the two-dimensional state into L_{B}^{est}.

As shown in FIG. 26, the estimation error of the degree of liberation (E) in a case of substituting the degree of liberation (L_{B}^{2D}) in the two-dimensional state is from 56.4% to 64.4%, whereas the estimation error of the degree of liberation (E) in a case of substituting the true value estimation data (L_{B}^{3D′}) for the degree of liberation of the estimation target in the three-dimensional state is from 13.0% to 16.4%. Through the estimation processing in the present invention, the estimation error of the degree of liberation (E) is reduced greatly.

(Indirect Estimation of True Value Estimation Data Using Correction Data)

Next, the contour line diagram shown in FIG. 19(b) set based on the multicomponent material formed of the spherical particles was used as the statistical data for collation with the fractal dimension value (δ) and the area fraction (Fa) of the multicomponent material with distorted particle shapes serving as the estimation target, which were input data, to read the degree-of-liberation over-estimation rate (σ_{B}) corresponding to the fractal dimension value (δ) and the area fraction (Fa) from the contour line diagram, and the degree-of-liberation over-estimation rate (σ_{B}) was set as the correction data (σ_{B′}) on the degree-of-liberation over-estimation rate for the multicomponent material formed of the distorted particles shapes.

The obtained correction data (σ_{B′}) on the degree-of-liberation over-estimation rate was used to correct the degree of liberation (L_{B}^{2D}) in the two-dimensional state in accordance with the following Equation (61) given below, and the true value estimation data (L_{B}^{3D″}) for the degree of liberation of the estimation target in the three-dimensional state was obtained.

[Numerical Equation 24]

L_{B}^{3D″}=(1−σ′_{b})L_{B}^{2D} (61)

The fractal dimension value (δ) and the area fraction (Fa) of the multicomponent material with distorted particle shapes, the correction data ( σ_{B′}), the true value estimation data (L_{B}^{3D″}) for the degree of liberation of the estimation target in the three-dimensional state, which was obtained through the correction, and the true value (L_{B}^{3D}) for the degree of liberation of the estimation target in the three-dimensional state, which was calculated from the virtual setting of the multicomponent material with distorted particle shapes, are illustrated in Table 2 shown below.

TABLE 2

| Type of | | | | | |
|---|---|---|---|---|---|
| particle | Fₐ | δ | σ′_{B} | L_{B}^{3D″} | L_{B}^{3D} |
| 1 | 0.151 | 2.213 | 0.380 | 0.396 | 0.408 |
| 2 | 0.151 | 2.213 | 0.380 | 0.396 | 0.407 |
| 3 | 0.151 | 2.215 | 0.380 | 0.395 | 0.399 |
| 4 | 0.151 | 2.217 | 0.390 | 0.386 | 0.393 |
| 5 | 0.151 | 2.215 | 0.380 | 0.396 | 0.408 |
| 6 | 0.151 | 2.215 | 0.380 | 0.396 | 0.407 |
| 7 | 0.151 | 2.217 | 0.390 | 0.387 | 0.401 |
| 8 | 0.152 | 2.220 | 0.400 | 0.377 | 0.387 |
| 9 | 0.152 | 2.219 | 0.390 | 0.387 | 0.401 |
| 10 | 0.152 | 2.219 | 0.390 | 0.387 | 0.401 |
| 11 | 0.152 | 2.219 | 0.390 | 0.386 | 0.395 |
| 12 | 0.152 | 2.222 | 0.400 | 0.377 | 0.382 |

Further, the true value estimation data (L_{B}^{3D″}) for the degree of liberation of the estimation target in the three-dimensional state, which was obtained through the correction, was substituted into L_{B}^{est} in the said Equation (60) to calculate the estimation error of the degree of liberation (E) for the true value (L_{B}^{3D}).

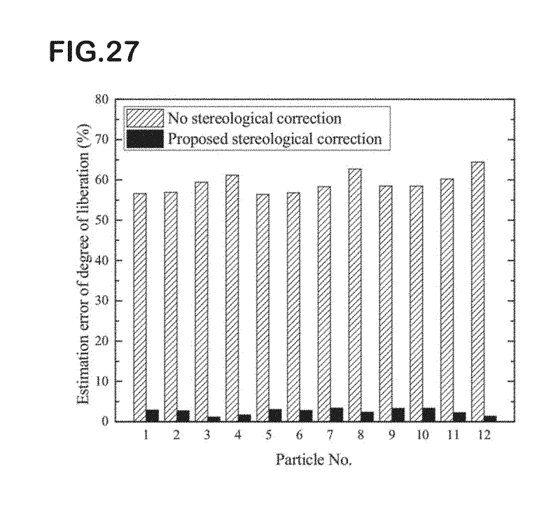

FIG. 27 is a graph for showing a comparison between the estimation error of the degree of liberation (E) for the true value (L_{B}^{3D}) that is obtained by substituting the true value estimation data (L_{B}^{3D″}) for the degree of liberation of the estimation target in the three-dimensional state, which was obtained thorough the correction, into L_{B}^{est} and the estimation error of the degree of liberation (E) for the true value (L_{B}^{3D}) that is obtained by substituting the degree of liberation (L_{B}^{2D}) of the estimation target in the two-dimensional state into L_{B}^{est}.

As shown in FIG. 27, the estimation error of the degree of liberation (E) in a case of substituting the degree of liberation (L_{B}^{2D}) in the two-dimensional state is from 56.4% to 64.4%, whereas the estimation error of the degree of liberation (E) in a case of substituting the true value estimation data (L_{B}^{3D″}) for the degree of liberation of the estimation target in the three-dimensional state, which was obtained through correction, is from 1.16% to 3.41%. Through the estimation processing in the present invention, the estimation error of the degree of liberation (E) is reduced greatly.

Further, the estimation error of the degree of liberation (E) for the true value estimation data (L_{B}^{3D′}) for the degree of liberation of the estimation target in the three-dimensional state is from 13.0% to 16.4% as described above, and correction is made through use of the correction data (σ_{B′}) on the degree-of-liberation over-estimation rate, to thereby obtain the true value estimation data for the degree of liberation of the estimation target in the three-dimensional state with higher estimation accuracy.

(Estimation of Degree of Locking)

Next, processing of estimating the degree of locking is performed in order to check the validity of the processing of estimating the degree of locking in the present invention.

In this description, indirect estimation of the true value estimation data using the correction data is adopted to estimate the degree of locking.

A multi-component particle (model particle) serving as the estimation target was set as follows.

Specifically, the shape of the model particle was set with a method similar to the method of setting the shape of the model particle described above so that the aspect ratio (α) was 2.0 and the corrected sphericity (S_{c}) was 0.95 for the spherical particle, which was the second-generation particle (refer to FIG. 20(b)) described with reference to FIG. 20.

Further, regarding the internal structure of the model particle, the core component was set in the model particle, and the phase A and the phase B were formed in the model particle with a method similar to the method of setting the internal structure of the model particle described above. Specifically, the phase A was set as the core component, and 9 patterns of the aspect ratio (δ) and the volume fraction (F_{V}) of the first-generation particle (refer to FIG. 23(b)) for setting of the core component were set as shown below in Table 3, to thereby set the internal structure of the model particle.

In this manner, 9 patterns of the spherical particle formed of two components, namely, the phase A and the phase B, were set as the model particle.

The degree of locking is estimated by focusing on the phase A.

TABLE 3

| | Aspect ratio (α) of particles of | Volume fraction (F_{V}) of |
|---|---|---|
| Case | core component | core component |
| 1 | 1.0 | 0.032 |
| 2 | 1.0 | 0.064 |
| 3 | 1.0 | 0.094 |
| 4 | 2.5 | 0.034 |
| 5 | 2.5 | 0.067 |
| 6 | 2.5 | 0.098 |
| 7 | 4.0 | 0.036 |
| 8 | 4.0 | 0.072 |
| 9 | 4.0 | 0.110 |

Next, respective contour line diagrams shown in FIG. 14(a) to FIG. 14(l) were set as the statistical data.

The respective contour line diagrams shown in FIG. 14(a) to FIG. 14(l) were created as the contour line diagram for the degree of locking in accordance with creation of the contour line diagrams shown in FIG. 18 and FIG. 19.

Next, the two-dimensional degree of locking (Λ_{A}^{2D}), the fractal dimension (δ), and the area fraction (Fa), which had been obtained in accordance with the above-mentioned method of calculating information on the 20 cross-sections for the 9 patterns of the model particles, were set as the estimation target data for collation with the statistical data, and the correction data and the three-dimensional state data were derived.

The correction data and the three-dimensional state data were derived at the time of setting of the statistical data by calculating an estimation value (Λ_{A}^{3D′}) of the three-dimensional degree of locking serving as the three-dimensional state data from the stereological bias corrected value (Λ_{A}^{Dif}) organized into statistics in accordance with the following Equation (62). In the following Equation (62), a supersubscript, for example, [0] is omitted.

[Numerical Equation 25]

Λ_{A}^{3D}′=Λ_{A}^{2D}−Λ_{A}^{Dif} (62)

Four error indicators, namely, an uncorrected area error (E₁), a corrected area error (E₁^{′}), an uncorrected maximum error (E₂), and a corrected maximum error (E₂^{′}) are defined in order to verify the validity of the processing of estimating the degree of locking in the present invention.

The uncorrected area error (E₁) indicates a difference in area between the two-dimensional degree of locking indicated by “Λ_{A}^{2D}” and the three-dimensional degree of locking indicated by “Λ_{A}^{3D}” in the graph for showing the distribution of the degree of locking illustrated in FIG. 12.

Further, similarly, the corrected area error (E₁′) indicates a difference in area between the estimation value (Λ_{A}^{3D′}) of the three-dimensional degree of locking and the three-dimensional degree of locking indicated by “Λ_{A}^{3D}”.

Further, the uncorrected maximum error (E₂) indicates the maximum interval between the two-dimensional degree of locking indicated by “Λ_{A}^{2D}” and the three-dimensional degree of locking indicated by “Λ_{A}^{3D}” in a vertical-axis direction in the graph for showing the distribution of the degree of locking shown in FIG. 12.

Further, similarly, the corrected maximum error (E₂^{′}) indicates the maximum interval between the estimation value (Λ_{A}^{3D′}) of the three-dimensional degree of locking and the three-dimensional degree of locking indicated by “ΛA^{3D}” in the vertical-axis direction.

Further, the area error improvement rate (I₁) and the maximum error improvement rate (I₂) are defined in accordance with the following Equations (63) and (64) given below in order to quantify the correction effect.

$$\begin{matrix}[ Numerical Equation 26 ] & \\ {I}_{1} = 100 \frac{{E}_{1} - {E}_{1}^{′}}{{E}_{1}} & ( 63 ) \\ {I}_{2} = 100 \frac{{E}_{2} - {E}_{2}^{′}}{{E}_{2}} & ( 64 )\end{matrix}$$

The area error improvement rate (I₁) and the maximum error improvement rate (I₂) are indicators for indicating a ratio of decrease of the originally existing stereological bias through correction, and thus it means that, as the values of the area error improvement rate (I₁) and the maximum error improvement rate (I₂) become larger, the stereological bias is reduced more.

The test result is shown in Table 4 below.

TABLE 4

| Case | E₁ | E₁′ | I₁ (%) | E₂ | E₂′ | I₂ (%) |
|---|---|---|---|---|---|---|
| 1 | 0.0131 | 0.00291 | 77.9 | 0.258 | 0.0222 | 91.4 |
| 2 | 0.0227 | 0.00466 | 79.4 | 0.349 | 0.0239 | 93.2 |
| 3 | 0.0307 | 0.00607 | 80.2 | 0.332 | 0.0541 | 83.7 |
| 4 | 0.00590 | 0.00121 | 79.6 | 0.0676 | 0.00264 | 96.1 |
| 5 | 0.0111 | 0.00235 | 78.8 | 0.126 | 0.00495 | 96.1 |
| 6 | 0.0158 | 0.00285 | 81.9 | 0.169 | 0.00358 | 97.9 |
| 7 | 0.00524 | 0.00167 | 68.1 | 0.0391 | 0.00581 | 85.1 |
| 8 | 0.00937 | 0.00232 | 75.3 | 0.0741 | 0.00369 | 95.0 |
| 9 | 0.0125 | 0.00194 | 84.5 | 0.103 | 0.00355 | 96.5 |

As shown in Table 4, it is confirmed that the area error improvement rate (I₁) indicates a large value of about 80% on average and the maximum error improvement rate (I₂) indicates a large value of about 90% on average through the processing of estimating the 9 patterns of the model particles in the present invention.

Therefore, with the estimation processing in the present invention, it is possible to greatly reduce the stereological bias, and obtain an excellent estimation result also for the three-dimensional state data on the degree of locking.

### REFERENCE SIGNS LIST

1, 10 statistical data setting means

2, 20 three-dimensional state estimation means

21 correction data deriving module

22 two-dimensional state data correction module

1. A three-dimensional state estimation device, comprising: statistical data setting means for setting statistical data obtained by taking statistics of a correlation among:
a complexity indicator for quantitatively indicating, as an image complexity, various display states of a component of interest in two-dimensional image data in which a cross-section or surface of a multicomponent material comprising a group of particles formed of a liberated particle and a locked particle is displayed and the component of interest and a component of non-interest in the group of particles in the cross-section or surface are displayed differently from each other;
an area fraction of the component of interest in the two-dimensional image data; and
three-dimensional estimation data, which is any one of three-dimensional state data on a content percentage of the component of interest in the multicomponent material at a time when the complexity indicator and the area fraction are determined and correction data for correcting two-dimensional state data on the content percentage of the component of interest in the cross-section or surface of the multicomponent material to the three-dimensional state data.

2. The three-dimensional state estimation device according to claim 1,
wherein the three-dimensional estimation data used for setting the statistical data by the statistical data setting means is the three-dimensional state data, and
wherein the three-dimensional state estimation device further comprises first three-dimensional state estimation means for deriving, when the complexity indicator and the area fraction of the multicomponent material serving as an estimation target are input, the three-dimensional state data corresponding to the input of the complexity indicator and the area fraction through collation with the statistical data set in the statistical data setting means, and capable of directly outputting the derived three-dimensional state data as true value estimation data for estimating a three-dimensional state of the estimation target.

3.The three-dimensional state estimation device according to claim 1,
wherein the three-dimensional estimation data used for setting the statistical data by the statistical data setting means is the correction data, and
wherein the three-dimensional state estimation device further comprises second three-dimensional state estimation means comprising:
a correction data deriving module configured to collate input of the complexity indicator and the area fraction of the multicomponent material serving as an estimation target with the statistical data set in the statistical data setting means, to thereby derive the correction data corresponding to the input of the complexity indicator and the area fraction; and
a two-dimensional state data correction module configured to correct the input two-dimensional state data on the multicomponent material serving as the estimation target through use of the correction data derived by the correction data deriving module, to thereby derive the three-dimensional state data, and capable of outputting the derived three-dimensional state data as true value estimation data for estimating a three-dimensional state of the estimation target.

4. The three-dimensional state estimation device according to claim 1, wherein the complexity indicator comprises any one of a fractal dimension value and a statistical feature calculated due to a difference in the image density value when different image density values are given to the component of interest and the component of non-interest.

5. The three-dimensional state estimation device according to claim 4, wherein the fractal dimension value δ is calculated in accordance with Equation (1) given below,
$$\begin{matrix}[ Numerical Equation 1 ] & \\ δ = 2 - \frac{log A ( r ) - C}{log r} & ( 1 )\end{matrix}$$
where:
r indicates a length of one side of a defined square region, which is defined by equally dividing a square region having a length of one side being R in the two-dimensional image data into N² blocks by any integer N;
A(r) indicates, when respective vertices of a square in the defined square region are denoted by A, B, C, and D, plane coordinates X and Y are set in the same plane as a plane of the vertices A, B, C, and D, and respective points set depending on image strengths at the respective vertices A, B, C, and D in the two-dimensional image data as a height Z in a direction orthogonal to the plane forming the plane coordinates X and Y are denoted by set points A′, B′, C′, and D′, a sum of areas of two triangles comprising one triangle having the set points A′, B′, and D′ as vertices, and another triangle having the set points B′, C′, and D′ as vertices, which are calculated for all the defined square regions in the square region; and
C indicates log A(1).

6. The three-dimensional state estimation device according to claim 4, wherein the three-dimensional state estimation device is configured to calculate the statistical feature through use of a density co-occurrence matrix P(i ,j:d,θ), which is a matrix indicating, when an entire or partial region of the two-dimensional image data represented by two or more tones of the density level is observed with XY plane coordinates, a frequency in the entire or partial region of a pair of a pixel 1 with a pixel density value of i and a pixel 2 with a pixel density value of j, which are any two pixels in the entire or partial region, where d represents a coordinate distance between the pixel 1 and the pixel 2 and θ represents an angle formed by a straight line connecting the two pixels and an X axis.

7. The three-dimensional state estimation device according to claim 4, wherein the three-dimensional state estimation device is configured to calculate the statistical feature through use of a density difference vector Q(i:d,θ), which is a vector indicating, when an entire or partial region of the two-dimensional image data represented by two or more tones of the density level is observed with XY plane coordinates, a frequency in the entire or partial region of a pair of a pixel 1 and a pixel 2, which are any two pixels in the entire or partial region, with a difference between a pixel density value of the pixel 1 and pixel density value of the pixel 2 being i, where d represents a coordinate distance between the pixel 1 and the pixel 2 and θ represents an angle formed by a straight line connecting the two pixels and an X axis.

8. A The three-dimensional state estimation device according to any claim 1, wherein the three-dimensional state data comprises a degree of liberation indicating any one of an area fraction, a volume fraction, a mass fraction, and a count fraction of a liberated particle in the group of particles.

9. The three-dimensional state estimation device according to claim 1, wherein the three-dimensional state data comprises a degree of locking indicating any one of an area fraction, volume fraction, mass fraction, and count fraction in a group of locked particles in which any one of an area fraction, volume fraction, and mass fraction of a component of interest in one particle is a fixed fraction.

10. A three-dimensional state estimation program for causing a computer to function as statistical data setting means for setting statistical data obtained by taking statistics of a correlation among:
a complexity indicator for quantitatively indicating, as an image complexity, various display states of a component of interest in two-dimensional image data in which a cross-section or surface of a multicomponent material comprising a group of particles formed of a liberated particle and a locked particle is displayed and the component of interest and a component of non-interest in the group of particles in the cross-section or surface are displayed differently from each other;
an area fraction of the component of interest in the two-dimensional image data; and
three-dimensional estimation data, which is any one of three-dimensional state data on a content percentage of the component of interest in the multicomponent material at a time when the complexity indicator and the area fraction are determined and correction data for correcting two-dimensional state data on the content percentage of the component of interest in the cross-section or surface of the multicomponent material to the three-dimensional state data.

11. A three-dimensional state estimation method, comprising a statistical data setting step of setting statistical data obtained by taking statistics of a correlation among:
a complexity indicator for quantitatively indicating, as an image complexity, various display states of a component of interest in two-dimensional image data in which a cross-section or surface of a multicomponent material comprising a group of particles formed of a liberated particle and a locked particle is displayed and the component of interest and a component of interest in the group of particles in the cross-section or surface are displayed differently from each other;
an area fraction of the component of interest in the two-dimensional image data; and
three-dimensional estimation data, which is any one of three-dimensional state data on a content percentage of the component of interest in the multicomponent material at a time when the complexity indicator and the area fraction are determined and correction data for correcting two-dimensional state data on the content percentage of the component of interest in the cross-section or surface of the multicomponent material to the three-dimensional state data.
